(19) United States
(12) Patent Application Publication
(10) Pub. No.: US 2007/0122903 A1
(43) Pub. Date: May 31, 2007

(54) AMNIOTIC FLUID DERIVED CELLS

(76) Inventors: Alireza Rezania, Hillsborough, NJ (US); Jean Xu, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/420,895

(22) Filed: May 30, 2006

Related U.S. Application Data
(60) Provisional application No. 60/685,607, filed on May 27, 2005.
(60) Provisional application No. 60/743,821, filed on Mar. 27, 2006.

(51) Int. Cl. C12N 5/06; C12N 5/08

(52) U.S. Cl. 435/325; 435/368

(57) ABSTRACT

This invention relates to an expandable population of amniotic fluid-derived cells that can be differentiated into a β-cell lineage. This invention also provides methods for isolating and expanding such amniotic fluid-derived cells, as well as related methods and compositions for utilizing such cells in the therapeutic treatment of diabetes.

Related U.S. application data: Provisional application No. 60/685,607, filed May 27, 2005. Provisional application No 60/743,821, filed Mar. 27, 2006.

### FIELD OF THE INVENTION

This invention relates to an expandable population of amniotic fluid-derived cells that can be differentiated into a β-cell lineage. This invention also provides methods for isolating and expanding such amniotic fluid-derived cells, as well as related methods and compositions for utilizing such cells in the therapeutic treatment of diabetes.

### BACKGROUND

Loss of organ function can result from congenital defects, injury or disease. One example of a disease causing loss of organ function is diabetes mellitus, or diabetes. Most cases of diabetes fall into two clinical types: Type 1, also known as juvenile-onset diabetes, or insulin dependent diabetes mellitus (IDDM), and Type 2, also known as adult-onset diabetes. Each type has a different prognosis, treatment, and cause. Both types are characterized by the patient's inability to regulate their blood glucose levels. As a consequence, blood glucose levels rise to high values because glucose cannot enter cells to meet metabolic demands. This inability to properly metabolize blood sugar causes a complex series of early and late-stage symptomologies, beginning with, for example, hyperglycemia, abnormal hunger, thirst, polyuria, and glycouria, and then escalating to, for example, neuropathy, macro-vascular disease, and micro-vascular disease.

A common method of treatment of Type 1 diabetes involves the exogenous administration of insulin, typically by injection with either a syringe or a pump. This method does not completely normalize blood glucose levels and is often associated with an increased risk of hypoglycemia. More effective glycemic control can be achieved if the function of the pancreas can be restored or rejuvenated via transplantation or cell-based therapies.

There are many transplantation therapies currently used to treat diabetes: One such treatment involves transplanting isolated islets of Langerhans into the diabetic patient. One of the main hurdles to human islet transplantation has been the lack of sufficient number of islets to treat the large number of diabetic patients. One possible solution to the shortage of islets is the generation of islets from alternate cellular sources.

It has been documented that progenitor cells derived from adult tissues are capable of differentiation into a pancreatic β-cell phenotype. See, for example, WO2004/087885 A2, Hess et al. (Nature Biotechnology 21, 763-770, 2003), and Ianus et al. (J. Clin. Invest. 111: 843-850, 2003), which report the capacity of adult bone marrow-derived cells (mesenchymal and hematopoetic cells) to differentiate into cells having characteristics of a pancreatic β-cell in vitro, or secrete trophic factors that help regenerate a damaged pancreas in vivo.

Among other sources of progenitor cells that can be differentiated into pancreatic cells include rodent liver oval stem cells (WO03/033697) and post-partum placenta (U.S. Published Application 2004/0161419 A1).

The endocrine cells of the islets of Langerhans, including β-cells, are constantly turning over by processes of apoptosis and the proliferation of new islet cells (neogenesis). As such, the pancreas is thought to be a source of progenitor cells that are capable of differentiating into pancreatic hormone producing cells. There are three distinct tissue types, isolated from a pancreas, that are a potential source of pancreatic progenitor cells: an islet rich fraction, a ductal cell rich fraction, and an acinar cell rich fraction.

Isolation of progenitor cells or partially differentiated cells from crude pancreatic tissue extracts may be achieved using antibodies raised against cell surface markers. For example, U.S. Published Application 2004/0241761 discloses isolation of murine cells that expressed ErbB2, ErbB3, ErbB4, Msx-2, PDX-1 and insulin. Gershengorn et al. (Science 306: 2261-2264, 2004) teach the production of proliferating cells that were able to form islet-like cell aggregates. The cells were derived from a heterogeneous population of adherent cells that emerged from the culture of isolated human pancreatic islets in vitro. The isolated islets of Langerhans were initially seeded onto tissue culture dishes and cultured in medium containing 10% serum. Fibroblast-like cells were observed to migrate out of the cultured islets and form a monolayer. These cells expressed Nestin, smooth muscle actin and vimentin.

Pancreatic progenitor cells may also arise from the culture of pancreatic islet and ductal tissue that has been dissociated into single cells, as disclosed by Seaberg et al. (Nature Biotechnology 22: 1115-1124, 2004). The murine progenitor cells disclosed by Seaberg et al. expressed Nestin during proliferation.

U.S. Published Application 2003/0082155 discloses methods to isolate and identify a population of cells from the islets of Langerhans of human pancreas, which have the functional and molecular characteristics of stem cells. In particular, these cells were characterized by Nestin-positive staining, Nestin gene expression, GLP-1R-positive staining, GLP-1R gene expression, ABCG2 positive staining, ABCG2 gene expression, Oct3/4 positive staining, Oct3/4 gene expression, latrophilin (type 2) positive staining, latrophilin (type 2) gene expression, Hes-1 positive staining, Hes-1 gene expression, Integrin subunits α6 and β1 positive staining, Integrin subunits α6 and β1 gene expression, c-kit positive staining, c-kit gene expression, MDR-1 positive staining, MDR-1 gene expression, SST-R, 2, 3, 4 positive staining, SST-R, 2, 3, 4 gene expression, SUR-1 positive staining, SUR-1 gene expression, Kir 6.2 positive staining, Kir 6.2 gene expression, CD34 negative staining, CD45 negative staining, CD133 negative staining, MHC class I negative staining, MHC class II negative staining, cytokeratin-19 negative staining, long-term proliferation in culture, and the ability to differentiate into pseudo-islets in culture.

In another approach, as disclosed in U.S. Pat. No. 5,834,308, U.S. Pat. No. 6,001,647 and U.S. Pat. No. 6,703,017, crude preparations of islet cultures from NOD mice may be used to establish epithelial-like cultures, which can be maintained in growing cultures for greater than 1 year and which appear to demonstrate the ability to differentiate into islet-like clusters, capable of secreting insulin.

Islet-like structures may be generated from fractions of digested human pancreata enriched for ductal tissue, as disclosed in Bonner-Weir et al. (Proc Nat Acad Sci 97: 7999-8004, 2000) and U.S. Pat. No. 6,815,203 B1. Islet-like clusters disclosed in these publications stained positive for cytokeratin-19 and showed immunoreactivity for insulin.

WO2004/011621 discloses the generation of insulin negative adherent cells from human pancreatic ductal fragments.

WO03/102134 discloses the generation of an epithelial cell positive for cytokeratin-19 from an acinar fraction of a human pancreatic digest. The cells generated are capable of limited expansion and differentiate into an insulin-producing cell in the presence of an induction media.

U.S. Published Application 2004/015805 A1 reports that a subset of human pancreatic stem cells may be isolated using ligands to the cell surface marker CD56 (also known as NCAM). These cells can differentiate into insulin producing cells and insulin producing aggregates.

It has been documented that progenitor cells, derived from fetal or embryonic tissues, have the potential to differentiate into a pancreatic hormone-producing cell. See, for example, U.S. Pat. No. 6,436,704, WO03/062405, WO02/092756 and EP 0 363 125 A2, which report the potential of human fetal and embryonic derived cells to differentiate into a β-cell lineage.

Human Embryonic Stem cells (hES) are derived from the inner cell mass of the blastocyst, the earliest stage of embryonic development of the fertilized egg. The blastocyst is a pre-implantation stage of the embryo, a stage before the embryo would implant in the uterine wall. When cultured on an inactivated feeder layer of cells according to conditions described by Thompson and colleagues (Thomson, et al. (Proc. Natl. Acad. Sci. U.S.A. 92: 7844-7848, 1995); Thomson, et al (Science 282:1145-1147, 1998), Marshall, et al., (Methods Mol. Biol. 158:11-18, 2001), the inner layer cells of the blastocyst may be grown in vitro indefinitely in an undifferentiated state. Properly propagated hES cells have unlimited potential to double while maintaining their pluiripotency; namely their capacity of differentiating into the three layers of the embryo, Ectoderm (Ec), Mesoderm (Me) and Endoderm (En). When grown as pluripotent hES, the cells maintain a euploid karyotype and are not prone to senescence.

Human embryonic stem cells display a distinct group of cell surface antigens, SSEA-3, SSEA-4, TRA-2-54 (alkaline phosphatase), TRA-1-60 and TRA-1-81, in addition to expressing specific transcription factors OCT-4, NANOG, SOX-2, FGF-4 and REX-1 (Henderson, et al., (Stem Cells 20:329-337, 2002), Draper, et al., (J. Anat. 200:249-258, 2002), Mitsui et al., (Cell 113:631-642, 2003), Chambers et al., (Cell 113:643-655, 2003).

It is important to note from these publications, however, that human embryonic cells often require a feeder layer for expansion and maintenance of pluripotency or combination of a complex extracellular matrix, such as, for example, MATRIGEL™, plus conditioned media. These conditions do not allow the facile scale up of cells and an eventual cell therapy for treating diabetes.

Researchers have found that non-embryonic types of stem cells (“adult stem cells”) are not as capable of differentiating into many different tissue types, as are embryonic stem cells, so embryonic stem cells still have many advantages over the use of adult stem cells. However, one obstacle with the isolation of embryonic stem cells is that the cells are derived from embryos at the “blastocyst” stage. Human embryonic stem cell research is encumbered by an emotionally charged political and ethics debate and is likely to remain so for years to come.

Additionally, human embryonic stem cells (hES) have been found to be tumorigenic when injected into immunologically impaired animals, i.e. in the context of post-natal tissues, whereas adult stem cells are not. The tumorigenic attributes of hES cells are not frequently addressed, though this issue may burden their use in replacement cell therapy in the future. The political, moral and ethical issues around hES cells and their tumorigenic properties, as well as the perceived difficulties of expanding undifferentiated adult stem cells in culture, while maintaining a genetically normal genome, are major barriers in the development of human cell replacement therapy.

Pluripotent or multipotent stem cells have been isolated from chorionic villus, and amniotic fluid. Many amniotic and placental cells share a common origin, namely the inner cell mass of the morula, which gives rise to the embryo itself, the yolk sac, the mesenchymal core of the chorionic villi, the chorion and the amnion (Crane & Cheung, Prenatal Diagnosis 8: 119-129, 1988). Embryonic and fetal cells from all three germ layers have long been identified in the amniotic fluid (Milunsky, Genetic Disorder of the Fetus. New York: Plenum Press, 75-84, 1979; Hoehn & Salk, Methods in Cell Biology 26, 11-34, 1982; Gosden, British Medical Bulletin 39, 348-354, 1983; Prusa et al, Human Reproduction 18, 1489-1493, 2003). Thus, amniotic fluid may provide the least invasive access to embryonic-like and fetal-like stem cells.

Amniotic fluid derived cells have been routinely used for detecting chromosomal abnormality of the fetus. Amniotic fluid is typically sampled during the 2nd trimester (16 to 22 weeks of gestation). Previous art clearly demonstrates presence of three sub-population with distinct cell morphologies: “fibroblastic” (F), “amniotic fluid” (AF) cells, and “epithelial” (E) cells. The F and AF cells rapidly expand whereas the E cells display a much slower growth curve and have poor clonal efficiency.

For example, PCT application WO2003/042405 discloses isolation of c-Kit positive stem cells from chorionic villus, amniotic fluid and placenta (Cell 1, Table I).

In another example, U.S. Published Application 2005/0054093 discloses the isolation of stem cells from amniotic fluid. These cells express stage-specific embryonic antigen 3 (SSEA3), stage-specific embryonic antigen 4 (SSEA4), Tra1-60, Tra1-81, Tra2-54, Oct-4, HLA class I, CD13, CD44 CD49b and CD105 (Cell 2, Table I).

In another example, fetal cells have been isolated from amniotic fluid (in't Anker et al, Blood 102, 1548-1549, 2003). The cells disclosed were positive for expression of the following markers: CD44, CD73, CD90, CD105, CD106, HLA-A, B, & C. The cells were negative for expression of the following markers: c-Kit (CD117), CD11, CD31, CD34, CD45 and HLA-D (Cell 3, Table I).

A population of mesenchymal stem cells isolated from amniotic fluid has also been reported in a publication to Tsai et al (Tsai et al, Human Reproduction 19, 1450-1456, 2004). The cells disclosed were positive for expression of the following markers: CD29, CD44, CD73, CD90, HLA-A, B, & C. The cells were also positive for the embryonic transcription factor Oct-4. The cells were negative for expression of the following markers: c-Kit (CD117), CD34 and HLA-D (Cell 4, Table I).

Although recent publications and patents have suggested that within the fibroblastic, amniotic fluid, or epithelial subpopulations there exists a cell population that display some characteristics of human embryonic cells, such as expression of surface markers SSEA3 and -4, expression of transcription factor Oct-4, strong expansion potential, and differentiation into multiple cell types; none of the previously published art has demonstrated the existence of a subpopulation of the cells that display expression of key early endodermal markers, such as HNF-1 beta, HNF-3 beta, SOX-17, and GATA-6, while maintaining expression of ES markers SSEA-4.

Co expression of HNF-1 beta, HNF-3 beta (also known as FOXa2), SOX-17, and GATA-6 is regarded as the key step to define the formation of definitive endoderm during gastrulation. Thus, expression of these markers may be key in the generation of a pancreatic β-cell population, or a population of pancreatic hormone-producing cells, or a gut hormone-producing cell from an amniotic fluid-derived cell.

Therefore, there still remains a significant need to develop culture conditions for establishing amniotic fluid-derived cell lines that can be expanded to address the current clinical needs, while retaining the potential to differentiate into definitive endoderm, or a population of pancreatic hormone-producing cells, or a gut hormone-producing cell, or a β-cell lineage.

### SUMMARY

In one embodiment, the present invention provides a method for isolating mammalian amniotic fluid-derived cells. According to the present invention, amniotic fluid-derived cells are obtained from amniotic fluid samples of about 14 to about 23 weeks gestation. Alternatively, the amniotic fluid-derived cells are obtained from amniotic fluid samples of about 23 to about 40 weeks gestation.

In one embodiment, the cultures are left undisturbed for at least 5 to 10 days under hypoxic conditions (3% O₂). Alternatively, the cultures are left undisturbed for at least 5 to 10 days under normoxic conditions (approximately 20% O₂).

In an alternate embodiment, amniotic fluid-derived cells are obtained from amniotic fluid samples from the second trimester of gestation. Alternatively, the amniotic fluid-derived cells are obtained from amniotic fluid samples from the third trimester of gestation.

In one embodiment, the cultured amniotic fluid-derived cells are isolated as single cells, and clonally expanded.

The amniotic fluid-derived isolated according to the methods of the present invention can be contacted, for example, with an agent (such as an antibody) that specifically recognizes a protein marker expressed by amniotic fluid cells, to identify and select amniotic fluid-derived cells, thereby obtaining a substantially pure population of amniotic fluid-derived cells, i.e., wherein a recognized protein marker is expressed in at least 50% of the cell population.

In one embodiment, the resulting amniotic fluid-derived cell population is substantially positive for at least one of the following markers: HNF-1 beta, HNF-3 beta, SOX-17, or GATA-6. The amniotic fluid-derived cell population is substantially negative for at least one of the following markers: CD117, Oct-4, or Tra2-54. The amniotic fluid-derived cell population can be expanded for more than 50 population doublings without losing the capacity to express HNF-1 beta, HNF-3 beta, SOX-17, or GATA-6.

In one embodiment, the amniotic fluid-derived cell population is substantially positive for the following markers: SSEA4 and CD44. The amniotic fluid-derived cell population can be expanded for more than 50 population doublings without losing the capacity to express HNF-1 beta, HNF-3 beta, SOX-17, or GATA-6.

In one embodiment, the amniotic fluid-derived cell population isolated according to the methods of the present invention is substantially negative for at least one of the following markers: SOX-17, CD117, Oct-4, or Tra2-54. The amniotic fluid-derived cell population is substantially positive for the following markers: SSEA4 and CD44. The amniotic fluid-derived cell population can be expanded for more than 50 population doublings.

In one embodiment, the amniotic fluid-derived cell population isolated according to the methods of the present invention is substantially negative for cytokeratin and at least one of the following markers: SOX-17, CD117, Oct4, or Tra2-54. The amniotic fluid-derived cell population is substantially positive for the following markers: SSEA4 and CD44. The amniotic fluid-derived cell population can be expanded for more than 50 population doublings.

In one embodiment, the amniotic fluid-derived cell population isolated according to the methods of the present invention is substantially negative for SOX-17. The amniotic fluid-derived cell population is substantially positive for the following markers: SSEA4 and CD44. The amniotic fluid-derived cell population can be expanded for more than 50 population doublings.

In one embodiment, the amniotic fluid-derived cell population isolated according to the methods of the present invention is substantially negative for the following markers: cytokeratin, and SOX-17. The amniotic fluid-derived cell population is substantially positive for the following markers: SSEA4 and CD44. The amniotic fluid-derived cell population can be expanded for more than 50 population doublings.

In one embodiment, the amniotic fluid-derived cell population isolated according to the methods of the present invention is substantially negative for SOX-17. The amniotic fluid-derived cell population is further negative for at least one of the following markers: CD117, Oct4, or Tra2-54. The amniotic fluid-derived cell population is substantially positive for the following markers: SSEA4 and CD44. The amniotic fluid-derived cell population can be expanded for more than 50 population doublings.

In one embodiment, the amniotic fluid-derived cell population isolated according to the methods of the present invention is substantially negative for the following markers: cytokeratin, and SOX-17. The amniotic fluid-derived cell population is further negative for at least one of the following markers: CD117, Oct4, or Tra2-54. The amniotic fluid-derived cell population is substantially positive for the following markers: SSEA4 and CD44. The amniotic fluid-derived cell population can be expanded for more than 50 population doublings.

In another embodiment, the present invention provides an isolated pure population of amniotic fluid-derived cells that are substantially negative for at least one of the following markers: CD117, Oct4, or Tra2-54.

In another embodiment, the present invention provides an isolated pure population of amniotic fluid-derived cells that are substantially negative for at least one of the following markers: SOX-17, CD117, Oct4, or Tra2-54.

In another embodiment, the present invention provides an isolated pure population of amniotic fluid-derived cells that are substantially negative for SOX-17.

In another embodiment, the present invention provides an isolated pure population of amniotic fluid-derived cells that are substantially negative for SOX-17, and substantially negative for at least one of the following markers: CD117, Oct4, or Tra2-54.

In one embodiment, the amniotic fluid-derived cells isolated according to the methods of the present invention may also express at least one of the following: Musashi-1 and Hes1.

The amniotic fluid-derived cells isolated and expanded according to the present invention can be induced to differentiate into cells of the β cell lineage under appropriate in vitro or in vivo conditions. Accordingly, the amniotic fluid-derived cells selected and expanded according to the present invention, as well as the differentiated cells derived from the amniotic fluid-derived cells, are useful for treating Type 1 and 2 diabetes.

The amniotic fluid-derived cells isolated and expanded according to the present invention can be induced to gut hormone-producing cells under appropriate in vitro or in vivo conditions. In one embodiment, the amniotic fluid-derived cells isolated and expanded according to the present invention can be induced to gut hormone-producing cells under appropriate in vitro or in vivo conditions and may express insulin in a glucose responsive manner.

### BRIEF DESCRIPTION OF THE DRAWINGS

FIG. 1 shows the isolation and culturing steps used to isolate the amniotic fluid derived cells of the present invention.

FIG. 2 shows three distinct morphologies of cells isolated from an amniotic fluid sample at passage 0. a) AF morphology, b) epithelial morphology, and c) fibroblast morphology.

FIG. 3 depicts the expression of cell surface markers on AF-I cells derived from amniotic fluid. The markers are indicated on panels a-n.

FIG. 4 depicts the expression of cell surface markers on F cells derived from amniotic fluid. The markers are indicated on panels a-l.

FIG. 5 depicts the expression of cell surface markers on E cells derived from amniotic fluid. The markers are indicated on panels a-m.

FIG. 6 depicts immunofluoresence images of the F cells derived from amniotic fluid samples. F cells stained positive for a) vimentin, b) SSEA-4, and c) beta III tubulin.

FIG. 7 depicts immunofluoresence images of the E cells derived from amniotic fluid samples. E cells stained positive for a) vimentin and nestin, b) SSEA-4, c) beta III tubulin, d) pan-cytokeratin, e) smooth muscle actin, and f) cytokeratin 19.

FIG. 8 depicts immunofluoresence images of the AF-I cells derived from amniotic fluid samples. AF-I cells stained positive for a) vimentin and nestin, b) beta III tubulin, c) cytokeratin 19 and HES-1, d) pan-cytokeratin, e) SSEA-4, f) SOX-17 and ZO-1, g) GATA-6, h) HNF-1 beta, i) smooth muscle actin and HES-2.

FIG. 9 shows the expression profile of AF-I, AF-II, and AF-III cells of the present invention.

FIG. 10 depicts the population doubling curve of early passage AF-I cells.

FIG. 11 depicts the expansion potential of AF, F, or E cell derived from different donors. ♦ shows the cell number of amniotic fluid-derived cells with AF-I morphology obtained from amniotic fluid from one donor at 14-23 weeks gestation. Cells were cultured in media number 5 (Table II). ▴ shows the cell number of amniotic fluid-derived cells with AF-I morphology obtained from amniotic fluid from a second donor at 14-23 weeks gestation. Cells were cultured in media number 5 (Table II). ▪ shows the cell number of amniotic fluid-derived cells with F morphology obtained from amniotic fluid from a third donor at 14-23 weeks gestation. Cells were cultured in media number 15 (Table II). * shows the cell number of amniotic fluid-derived cells with F morphology obtained from amniotic fluid from a fourth donor at 14-23 weeks gestation. Cells were cultured in media number 16 (Table II). ● shows the cell number of amniotic fluid-derived cells with E morphology obtained from amniotic fluid from a donor at 14-23 weeks gestation. Cells were cultured in media number 5. + shows the cell number of amniotic fluid-derived cells with AF-II morphology obtained from amniotic fluid from a second donor at 14-23 weeks gestation. Cells were cultured in media number 5 (Table II). Δ shows the cell number of amniotic fluid-derived cells with AF-III morphology obtained from amniotic fluid from a second donor at 14-23 weeks gestation. Cells were cultured in media number 5 (Table II)

FIG. 12 depicts the telomere length of an AF-I cell line cultured either in AMNIOMAX or DM-LG+10% FBS at an intermediate passage level (approximately 40 population doublings). Lane 1 is the molecular weight ladder, lane 2 is the high telomere length control, lane 3 is the low telomere length control, lane 4 is amniotic fluid-derived cells from a donor at Passage 12, cultured in DMEM-LG+10% FBS, lane 5 is amniotic fluid-derived cells from the same donor at passage 12, cultured in media #5, and lane 6 is an embryonic carcinoma cell line (NTERA cells) that serves as a positive control.

FIG. 13 shows the karyotype of a) AF-I, b) AF-II, and c) AF-III cells cultured at passage 7-9 (approximately 30-35 population doublings).

FIG. 14 depicts the expansion potential of a single AF derived cell from one donor at term (approximately 38 weeks). Cells were cultured in media number 5 (Table II).

FIG. 15 depicts the scatter plot gene expression profiles between the different amniotic fluid cell types. The Pearson correlation coefficient for each plot is also listed.

FIG. 16 shows the effects of growth factors on gene expression in amniotic fluid-derived cells. Amniotic fluid-derived cells were obtained from a single donor and cultured for 12 days in conditioned media that was obtained from cultures of PANC-1 cells. The media was supplemented with the growth factors indicated. The levels of expression of HNF-3 beta and somatostatin were determined by real-time PCR. Human pancreas total RNA was included as a calibrator. Panel a shows the changes in HNF-3 beta expression. Panel b shows the changes in somatostatin expression.

FIG. 17 shows the effects of L685,458 on cultured amniotic fluid-derived cells having the AF morphology. Panel a shows the relative differences in RNA expression of human Hes-1 in cultured AF cells treated with the concentrations of L685,458 indicated. Panel b shows the effects of L685,458 on the viability of the cultured cells following a treatment with L685,458. Cells were treated for three days, at the concentrations indicated. Changes in viability, corresponding to cytotoxicity were detected using an MTS assay, where a decrease in cell viability corresponds to a decrease in A490 nm.

### DETAILED DESCRIPTION

For clarity of disclosure, and not by way of limitation, the detailed description of the invention is divided into the following subsections that describe or illustrate certain features, embodiments or applications of the present invention.

The present invention is directed to methods for isolating an amniotic fluid-derived cell population that is highly proliferative, and displays embryonic-like characteristics. Similar cells may also be present in the chorionic villus. Some of the embodiments of the invention disclosed herein describe three morphologically distinct populations of amniotic fluid-derived cells: “fibroblastic” (F), epithelial” (E) cells, and “amniotic fluid” (AF) cells.

### Definitions

“β-cell lineage” refer to cells with positive gene expression for the transcription factor PDX-1 and at least one of the following transcription factors: NGN-3, Nkx2.2, Nkx6.1, NeuroD, Isl-1, HNF-3 beta, MAFA, Pax4, and Pax6. Characteristics of cells of the beta cell lineage are well known to those skilled in the art, and additional characteristics of the beta cell lineage continue to be identified. These transcription factors are well established in prior art for identification of endocrine cells (Nature Reviews Genetics, Vol 3, 524-632, 2002).

“Pancreatic islet-like structure” refers to a three-dimensional clusters of cells derived by practicing the methods of the invention, which has the appearance of a pancreatic islet. The cells in a pancreatic islet-like structure express at least the PDX-1 gene and one hormone selected from the list glucagon, somatostatin, or insulin.

The term “hypoxic” refers to oxygen levels less than 20%, preferably less than 10%, and more preferably less than 5% but more than 1%.

The term “normoxia” refers to atmospheric oxygen levels of about 20%.

The term “substantially positive,” when used in connection with a population of cells with respect to the expression of certain marker (such as a membrane receptor, cytoplasmic or nuclear protein, or a transcription factor), means that the marker is present or expressed in at least about 50%, alternatively at least about 60%, and alternatively at least about 70%, of the total cell population.

The term “substantially negative,” when used in connection with a population of cells with respect to the expression of certain marker (such as a membrane receptor, cytoplasmic or nuclear protein, or a transcription factor), means that the marker is not present or expressed in at least about 70%, alternatively about 80%, alternatively about 90%, of the total cell population.

A “stem cell” as used herein refers to an undifferentiated cell that is capable of extensive propagation either in vivo or ex vivo and capable of differentiation to other cell types.

A “progenitor cell” refers to a cell that is derived from a stem cell by differentiation and is capable of further differentiation to more mature cell types. Progenitor cells typically have more restricted proliferation capacity as compared to stem cells.

“Expandable population” refers to the ability of an isolated cell population to be propagated through at least 50 or more cell divisions in a cell culture system.

By “undifferentiated cells,” when used in connection with cells isolated from a amniotic fluid, are meant a population of amniotic fluid-derived cells that are substantially negative for the expression of PDX-1, or insulin.

By “differentiated cells,” when used in connection with cells isolated from amniotic fluid, are meant a population of amniotic fluid-derived cells that are substantially positive for the expression of PDX-1, or insulin.

“Markers” as used herein, are nucleic acid or polypeptide molecules that are differentially expressed in a cell of interest. In this context, differential expression means an increased level of the marker for a positive marker, and a decreased level for a negative marker. The detectable level of the marker nucleic acid or polypeptide is sufficiently higher or lower in the cells of interest, compared to other cells, such that the cell of interest can be identified and distinguished from other cells, using any of a variety of methods known in the art.

“c-Kit” and “CD117” both refer to a cell surface receptor tyrosine kinase having a sequence disclosed in Genbank Accession No. X06182, or a naturally occurring variant sequence thereof (e.g., allelic variant).

“CD9” is also referred to as “Motility-related protein-1 (MRP-1)” and is a transmembrane glycoprotein that has been implicated in cell adhesion, motility, proliferation, and differentiation.

“CD10” is also referred to as “Common Acute Lymphocytic Leukemia Antigen (CALLA)”. CD10 is a cell surface enzyme with neutral metalloendopeptidase activity and it is expressed in lymphoblastic, Burkitt's, and follicular germinal center lymphomas and in patients with chronic myelocytic leukemia. It is also expressed on the surface of normal early lymphoid progenitor cells, immature B Cells within adult bone marrow and germinal center B Cells within lymphoid tissue. CD10 is also present on breast myoepithelial cells, bile canaliculi, fibroblasts, brush border of kidney and gut epithelial cells.

“CD44” is also referred to as “Hermes antigen” and is the main cell surface receptor for hyaluronan. This CD is primarily expressed in most cell types, except for tissues/cells such as hepatocytes, some epithelial cells, and cardiac muscle.

“CD49f” is also referred to as “a6 integrin” and “VLA-6,” and associates with integrin subunit beta 1 to bind laminin. CD49f is expressed primarily on epithelial cells, trophoblasts, platelets, and monocytes.

“CD73” is also referred to as “ecto-5′-nucleotidase” and is primarily expressed on a subset of —B and T cells, bone marrow stromal cells, various epithelial cells, fibroblasts, and endothelial cells. “CD90” is also referred to as “Thy-1” and is primarily expressed on hematopoietic stem cells, connective tissue cells, and various fibroblastic and stromal cells.

“SSEA-1” (Stage Specific Embryonic Antigen-1) is a glycolipid surface antigen present on the surface of murine teratocarcinoma stem cells (EC), murine and human embryonic germ cells (EG), and murine embryonic stem cells (ES).

“SSEA-3” (Stage Specific Embryonic Antigen-3) is a glycolipid surface antigen present on the surface of human teratocarcinoma stem cells (EC), human embryonic germ cells (EG), and human embryonic stem cells (ES).

“SSEA-4” (Stage Specific Embryonic Antigen-4) is a glycolipid surface antigen present on the surface of human teratocarcinoma stem cells (EC), human embryonic germ cells (EG), and human embryonic stem cells (ES).

“TRA1-60” is a keratin sulfate related antigen that is expressed on the surface of human teratocarcinoma stem cells (EC), human embryonic germ cells (EG), and human embryonic stem cells (ES).

“TRA1-81” is a keratin sulfate related antigen that is expressed on the surface of human teratocarcinoma stem cells (EC), human embryonic germ cells (EG), and human embryonic stem cells (ES).

“TRA2-49” is an alkaline phosphatase isozyme expressed on the surface of human teratocarcinoma stem cells (EC), and human embryonic stem cells (ES).

“Oct-4” is a member of the POU-domain transcription factor and is widely regarded as a hallmark of pluripotent stem cells. The relationship of Oct-4 to pluripotent stem cells is indicated by its tightly restricted expression to undifferentiated pluripotent stem cells. Upon differentiation to somatic lineages, the expression of Oct-4 disappears rapidly.

“EPCAM” “is also referred to as “Epithelial Cell Adhesion Molecule” is broadly expressed on cells of epithelial origin and epithelial derived tumor cells.

“Rex-1” is a developmentally regulated acidic zinc finger gene (Zfp-42). Rex-1 message level is high in embryonic stem cells and reduced upon induction of differentiation. As expected for a stem-cell-specific message, Rex-1 mRNA is present in the inner cell mass (ICM) of blastocyst, polar trophoblast of the blastocyst and later in the ectoplacental cone and extraembryonic ectoderm of the egg cylinder (trophoblast-derived tissues), but its abundance is much reduced in the embryonic ectoderm, which is directly descended from the ICM.

“HNF-1 alpha”, “HNF-1 beta” and “HNF-3 beta” belong to the hepatic nuclear factor family of transcription factors, which is characterized by a highly conserved DNA binding domain and two short carboxy-terminal domains.

“GATA-4” and “GATA-6” are members of the GATA transcription factor family. This family of transcription factors are induced by TGF β signaling and contribute to the maintenance of early endoderm markers, Sox 17α and HNF-1 beta, and the later marker HNF-3 beta.

“SOX-17” is a transcription factor, which is implicated in the formation of endoderm during embryogenesis.

By “basic defined cell culture medium” is meant a serum free or serum containing, chemically defined cell growth medium. Such medium includes, but is not limited to, Dulbecco's Modified Eagle's Medium (DMEM), alpha modified Minimum Essential Medium (alpha MMEM), Basal Medium Essential (BME), CMRL-1066, RPMI 1640, M199 medium, Ham's F10 nutrient medium, KNOCKOUT™ DMEM, Advanced DMEM, MCDB based media such as MCDB-151, -153, -201, and -302 (Sigma, Mo.), and DMEM/F12. These and other useful media are available from GIBCO, Grand Island, N.Y., U.S.A., for example. A number of these media are reviewed in Methods in Enzymology, Volume LVIII, “Cell Culture,” pp. 62-72, edited by William B. Jakoby and Ira H. Pastan, published by Academic Press Inc.

“Hes-1”, also known as “hairy/enhancer of split-1” is a transcription factor that may influence cell fate determination.

“Musashi-1” is a member of a subfamily of RNA binding proteins that are highly conserved across species. Musashi-1 expression is highly enriched in proliferative cells within the developing central nervous system, and may be a stem cell marker in intestinal cells.

“Pharmaceutical carrier” refers to a biodegradable or non-degradable porous or non-porous matrix that can act as a carrier for transplantation of mammalian cells.

“Transplantation” as used herein, can include the steps of introducing a cell or a population of cells or tissue into a mammal such as a human patient. “Transplantation” may also include incorporating cells or tissue into a pharmaceutical carrier, and implanting the carrier in a mammal such as a human patient.

### Isolation of Amniotic Fluid-Derived Cells

In one aspect of the present invention, amniotic fluid-derived cells are isolated by a multi-stage method, which essentially involves:
Isolation of amniotic fluid,
Centrifugation of the amniotic fluid, followed by removal of the supernatant,
Resuspending the cell pellet in growth medium,
Culturing the tissues and cells in a low oxygen environment,
Leaving the culture undisturbed for about 5 to 10 days without any media changes,
Isolation of distinct colonies using cloning rings,
Culturing the isolated colonies in growth media
Serial dilution cloning and identification of single cells that give rise to proliferating colonies, and
Culturing the clones in growth media.

In an alternate embodiment, amniotic fluid-derived cells are isolated by a multi-stage method, which essentially involves:
Isolation of amniotic fluid,
Centrifugation of the amniotic fluid, followed by removal of the supernatant,
Resuspending the cell pellet in growth medium,
Culturing the tissues and cells in a normoxic environment,
Leaving the culture undisturbed for about 5 to 10 days without any media changes,
Isolation of distinct colonies using cloning rings,
Culturing the isolated colonies in growth media,
Serial dilution cloning and identification of single cells that give rise to proliferating colonies, and
Culturing the clones in growth media.

The culture plates may be pre-coated with agents such as, for example, fibronectin, vitronectin, laminin, collagen, gelatin, thrombospondin, placenta extracts, MATRIGEL™, tenascin, human serum, or combinations thereof.

If desirable, the amniotic fluid may be exposed, for example, to an agent (such as an antibody) that specifically recognizes a protein marker expressed by amniotic fluid cells, to identify and select amniotic fluid-derived cells, thereby obtaining a substantially pure population of amniotic fluid-derived cells.

Amniotic fluid-derived cells may be cultured in AMNIOMAX™ complete medium (Invitrogen). Alternatively, the cells may be cultured in Chang B/C medium (Irvine Scientific). Alternatively, the cells may be cultured in low glucose DMEM, supplemented with insulin-transferrin-selenium-X (ITS-X, Invitrogen, CA), 2% fetal bovine serum (FBS), 1% penicillin/streptomycin (P/S)+25 ng/ml bFGF. Alternatively, the cells may be cultured in, DM-KNOCKOUT™ media (Invitrogen, CA), supplemented with 20% KNOCKOUT™ serum replacement (Invitrogen, CA), 10 ng/ml bFGF. Alternatively, the cells may be cultured in Williams' medium E supplemented with 2% defined FBS, 2 mM L-glutamine, ITS, 55 μM 2-mercaptoethanol, 10 ng/ml EGF, 4 ng/ml bFGF, and 4 ng/ml dexamethasone. Alternatively, the cells may be cultured in 1:1 DMEM-LG/MCDB 201, 2% FBS, ITS-X, βme 55 μM, 100 μM ascorbic acid-2-phosphate, 4 ng/ml bFGF, 10 ng/ml EGF, and 4 ng/ml dexamethasone. Alternatively, the cells may be cultured in low glucose DMEM, supplemented with 20% FBS. Alternatively, the cell may be cultured in low glucose DMEM, supplemented with 5% FBS. The cells may also be cultured in low glucose DMEM/MCDB 201 medium (1:1), supplemented with 2% defined FBS, ITS-X, 1 nM dexamethasone, 100 mM ascorbic acid 2-phosphate, 10 ng/ml EGF, 10 ng/ml PDGF-bb and 100 mM 2-mercaptoethanol. The media may be supplemented with bFGF, at concentrations from about 5 ng/ml to about 100 ng/ml. Alternatively, the cells may be cultured in 20% KNOCKOUT™ serum replacement+80% KNOCKOUT™ DMEM, supplemented with 1 mM L-glutamine, 1% non-essential amino acids and 0.1 mM 2-mercaptoethanol. The medium may be conditioned overnight, on human or murine embryonic fibroblasts, human bone marrow derived stromal cells, or human placenta derived cells and supplemented with 4 ng/ml bFGF. Alternatively, the cells may be cultured in high glucose DMEM, supplemented with 20% defined FBS with 0.1 mM 2-mercaptoethanol. Table II lists the various media formulations used to culture the amniotic fluid-derived cells of the present invention.

During culture in growth media, the cells may be cultured under hypoxic or normoxic conditions. Under hypoxic conditions, oxygen levels are lower than 20%, alternatively lower than 10%, alternatively lower than 5%, but more than 1%.

Preferably, the culture should be maintained in the growth media undisturbed for about 5 to 14 days without any media changes, at which point the cells have typically become adherent to the culture substrate used. At which point, cells may be sub-cultured.

Subculture can be achieved with any of the enzymatic solutions well known to those skilled in the art. An example of an enzymatic solution suitable for use in the present invention is TrypLE EXPRESS™ (Invitrogen, Ca).

Furthermore, the amniotic fluid-derived cells may be expanded by culturing in a defined growth media containing agent(s) that stimulate the proliferation of the cells of the present invention. These factors may include, for example, nicotinamide, members of TGF-β family, including TGF-β1, 2, and 3, bone morphogenic proteins (BMP-2, -4, 6, -7, -11, -12, and -13), serum albumin, fibroblast growth factor family, platelet-derived growth factor-AA, and -BB, platelet rich plasma, insulin growth factor (IGF-I, II) growth differentiation factor (GDF-5, -6, -8, -10, 11), glucagon like peptide-I and II (GLP-I and II), GLP-1 and GLP-2 mimetobody, Exendin-4, retinoic acid, parathyroid hormone, insulin, progesterone, testosterone, estrogen, aprotinin, hydrocortisone, ethanolamine, beta mercaptoethanol, epidermal growth factor (EGF), gastrin I and II, copper chelators such as triethylene pentamine, TGF-α, forskolin, Na-Butyrate, activin, betacellulin, noggin, neuron growth factor, nodal, insulin/transferring/selenium (ITS), hepatocyte growth factor (HGF), keratinocyte growth factor (KGF), bovine pituitary extract, islet neogenesis-associated protein (INGAP), proteasome inhibitors, notch pathway inhibitors, sonic hedgehog inhibitors, GSK-3 beta inhibitors, or combinations thereof. Alternatively, the amniotic fluid-derived cells may be expanded by culturing in conditioned media. By “conditioned media” is meant that a population of cells is grown in a basic defined cell culture medium and contributes soluble factors to the medium. In one such use, the cells are removed from the medium, while the soluble factors the cells produce remain. This medium is then used to nourish a different population of cells.

In certain embodiments, the amniotic fluid-derived cells are cultured on standard tissue culture plates. Alternatively, the culture plates may be coated with extracellular matrix proteins, such as, for example, MATRIGEL®, growth factor reduced MATRIGEL®, laminin, collagen, gelatin, tenascin, fibronectin, vitronectin, thrombospondin, placenta extracts, human serum, or combinations thereof.

### Characterization of the Isolated Amniotic Fluid-Derived Cells

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays, such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

Examples of antibodies useful for detecting certain protein markers are listed in Table III. It should be noted that other antibodies directed to the same markers that are recognized by the antibodies listed in Table III are available, or can be readily developed. Such other antibodies can also be employed for assessing expression of markers in the cells isolated in accordance with the present invention.

Characteristics of cells of the β-cell lineage are well known to those skilled in the art, and additional characteristics of the β-cell lineage continue to be identified. These characteristics can be used to confirm that the amniotic fluid-derived cells isolated in accordance with the present invention have differentiated to acquire the properties characteristic of the β-cell lineage. β-cell lineage specific characteristics include the expression of one or more transcription factors such as, for example, PDX-1(pancreatic and duodenal homeobox gene-1), NGN-3 (neurogenin-3), Hlxb9, Nkx6, Isl1, Pax6, NeuroD, Hnf1a, Hnf6, Hnf3 Beta, and Mafa, among others. These transcription factors are well established in the art for identification of endocrine cells. See, e.g., Edlund (Nature Reviews Genetics 3: 524-632 (2002)).

Characteristics of cells of the intestinal cell lineage are well known to those skilled in the art, and additional characteristics of this lineage continue to be identified. These characteristics can be used to confirm that the differentiated or undifferentiated amniotic fluid-derived cells isolated in accordance with the present invention have some of the properties characteristic of the intestinal cell lineage. Intestinal cell lineage characteristics include the expression of one or more transcription factors such as, for example, HES-1 (hairy/enhancer of split-1), NGN-3, Pax6, NeuroD, Math-1, and Musashi-1, among others. In addition, gut cells express hormones such as secretin, cholecystokinin, GLP-1, neurotensin, gastric inhibitory peptide (GIP), serotonin, somatostatin, and gastrin, among others. These transcription factors and gut hormones are well established in the art for identification of intestinal cells. See, e.g., Schonhoff (Endocrinology 145: 2639-2644 (2004)).

The present inventors have identified and isolated a population of amniotic fluid-derived cells that is highly proliferative, and displays embryonic cell-like characteristics, and may express at least one of the following markers: HNF-1 beta, HNF-3 beta, SOX-17, or GATA 6. In particular, the amniotic fluid-derived cells isolated in accordance with the present invention are characterized as, inter alia, substantially lacking at least one of the following protein markers: CD117, Oct-4 or Tra2-54. Populations of amniotic fluid-derived cells with these characteristics are referred to herein as AF-I (ATCC accession number PTA-6975).

Under the above growth conditions for expansion, the amniotic fluid cells isolated in accordance with the present invention may be expanded for more than 50 population doublings, while maintaining the potential to express at least one of the following markers: HNF-1 beta, HNF-3 beta, SOX-17, or GATA-6.

The present inventors have also identified and isolated populations of amniotic fluid-derived cells that is highly proliferative, displays embryonic cell-like characteristics, and do not express at least one of following markers: HNF-3 beta, SOX-17, GATA-4, CD117, Oct-4 or Tra2-54. In particular, the amniotic fluid-derived cells isolated in accordance with the present invention are characterized as, inter alia, substantially lacking at least one of the following protein markers: CD117, Oct-4 or Tra2-54. Populations of amniotic fluid-derived cells with these characteristics are referred to herein as AF-II.

The present inventors have also identified and isolated populations of amniotic fluid-derived cells that is highly proliferative, displays embryonic cell-like characteristics, and do not express any of the following markers: HNF-3beta, SOX-17, GATA-4, CD117, Oct-4 or Tra2-54. In particular, the amniotic fluid-derived cells isolated in accordance with the present invention are characterized as, inter alia, substantially lacking at least one of the following protein markers: CD117, Oct-4 or Tra2-54. Populations of amniotic fluid-derived cells with these characteristics are referred to herein as AF-II.

The present inventors have also identified and isolated populations of amniotic fluid-derived cells that is highly proliferative, displays embryonic cell-like characteristics, and do not express cytokeratin and at least one of following markers: HNF-3 beta, SOX-17, GATA-4, CD117, Oct-4 or Tra2-54. In particular, the amniotic fluid-derived cells isolated in accordance with the present invention are characterized as, inter alia, substantially lacking at least one of the following protein markers: CD117, Oct-4 or Tra2-54. Populations of amniotic fluid-derived cells with these characteristics are refered to herein as AF-III.

The present inventors have also identified and isolated populations of amniotic fluid-derived cells that is highly proliferative, displays embryonic cell-like characteristics, and do not express any of the following markers: cytokeratin, HNF-3beta, SOX-17, GATA-4, CD117, Oct-4 or Tra2-54. In particular, the amniotic fluid-derived cells isolated in accordance with the present invention are characterized as, inter alia, substantially lacking at least one of the following protein markers: CD117, Oct-4 or Tra2-54. Populations of amniotic fluid-derived cells with these characteristics are refered to herein as AF-III.

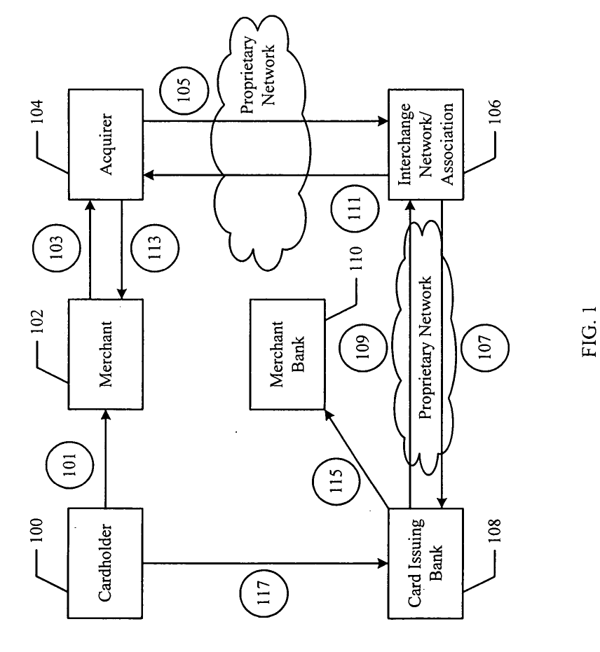

A summay of the expression profile of AF-I, AF-II and AF-III cells is shown in FIG. 9.

Amniotic fluid-derived cells of the present invention may be expanded for more than 50 population doublings, while maintaining the potential to differentiate into definitive endoderm, or cells with characteristics of a pancreatic β-cell lineage, or the capacity to differentiate into a gut hormone-producing cell.

### Differentiation of Amniotic Fluid-Derived Cells

In one aspect, the present invention provides compositions capable of differentiating the expanded amniotic fluid-derived cells of this invention into cells bearing markers characteristic of the β cell lineage.

In another aspect, the present invention provides compositions capable of differentiating the expanded amniotic fluid-derived cells of this invention into cells bearing markers characteristic of definitive endoderm.

In another aspect, the present invention provides compositions capable of differentiating the expanded amniotic fluid-derived cells of this invention into cells bearing markers characteristic of a gut hormone-producing cell.

A basic defined culture medium, when supplied with one or more components, that support the growth of amniotic fluid-derived cells, supplemented with differentiation-inducing amounts of one or more growth factors, is referred to as an “induction medium.” In accordance with the present invention, the induction medium contains less than or equal to 20% serum. In one embodiment, fetal calf serum may be used. Alternatively, fetal bovine serum may be replaced by serum from any mammal, or by albumin, bovine albumin or other compounds that permit or enhance differentiation of amniotic fluid-derived cells to the β cell lineage. Alternatively, the induction medium may be conditioned medium.

Factors appropriate for use in the induction medium may include, for example, nicotinamide, members of TGF-β family, including TGF-β1, 2, and 3, bone morphogenic proteins (BMP-2, -4, 6, -7, -11, -12, and -13), serum albumin, fibroblast growth factor family, platelet-derived growth factor -AA, and -BB, platelet rich plasma, insulin growth factor (IGF-I, II) growth differentiation factor (GDF-5, -6, -8, -10, 11), glucagon like peptide-I and II (GLP-I and II), GLP-1 and GLP-2 mimetobody, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as triethylene pentamine, TGF-α, forskolin, Na-Butyrate, activin, betacellulin, ITS, noggin, neurite growth factor, nodal, valporic acid, trichostatin A, sodium butyrate, hepatocyte growth factor (HGF), sphingosine-1, Wnt proteins such as Wnt-1, -3, -3a, 07a, and -8, keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, or combinations thereof.

In one aspect of the present invention, a combination of growth factors and chemical agents, including bFGF, Activin-A, FGF5, N2 and B27 supplements (Gibco, CA), steroid alkaloid such as, for example, cyclopamine (EMD, CA) that inhibits sonic hedgehog signaling, and a proteasome inhibitor such as, for example MG132 (EMD, CA), is supplied to a basic defined medium to support differentiation of amniotic fluid-derived cells into a β-cell lineage. In one aspect, the cells are cultured in an induction media composed of DMEM (low glucose, 5.5 mM) containing 10 micromolar MG-132 for 1-2 days, followed by additional incubation for 3-7 days in an induction media supplemented with 1×B27 (Gibco, CA) and 1×N2 (Gibco, CA) and further supplemented with Cyclopamine (10 μM; EMD, CA), bFGF (20 ng/ml; R&D Systems, MN), Activin A (20 nM; R&D Systems, MN) or FGF5 (20 ng/ml; R&D Systems, MN) for an additional five days.

In another aspect of the invention, the cells of the current invention can be treated with conditioned media isolated from cultures of primary fetal intestinal or pancreatic rudiments to induce further differentiation into the intestinal or pancreatic lineages, respectively. The cells may also be induce to differentiate with conditioned media from pancreatic cells lines such as PANC-1, CAPAN-1, BxPC-3, HPAF-II, hepatic cell lines such as HepG2, and intestinal cell lines such as, for example, FHs 74 and HS738. Alternatively, the cells of the present invention can be treated with conditioned media isolated from human or mouse embryonic stem cells indiced to differentiate into an endodermal lineage. These cell lines can be purchased from the ATCC (VA).

The combination and concentrations of growth factors, the length of culture, and other culture conditions can be optimized by those skilled in the art to achieve effective differentiation by, e.g., monitoring the percentage of cells that have differentiated into cells characteristic of the β-cell lineage. The one or more growth factors may be added in an amount sufficient to induce the differentiation of the amniotic fluid-derived cells of the present invention into cells bearing markers of a β-cell lineage over a time period of about one to four weeks.

### Therapeutic Use of the Cells of the Present Invention

In one aspect, the present invention provides a method for treating a patient suffering from, or at risk of developing Type 1 diabetes. This method involves isolating and culturing amniotic fluid-derived cells, expanding the isolated population of cells, differentiating the cultured cells in vitro into a β-cell lineage, and implanting the differentiated cells either directly or in a pharmaceutical carrier into the patient. If appropriate, the patient can be further treated with pharmaceutical agents or bioactives that facilitate the survival and function of the transplanted cells. These agents may include, for example, insulin, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, glucagon like peptide-I (GLP-1) and II, GLP-1 and 2 mimetibody, Exendin-4, retinoic acid, parathyroid hormone, MAPK inhibitors, such as, for example, compounds disclosed in U.S. Published Application 2004/0209901 and U.S. Published Application 2004/0132729.

In yet another aspect, this invention provides a method for treating a patient suffering from, or at risk of developing Type 2 diabetes. The method involves isolating and culturing amniotic fluid-derived cells according to the present invention, expanding the isolated population of cells, differentiating the cultured cells in vitro into a β-cell lineage, and implanting the differentiated cells either directly or in a pharmaceutical carrier into said patient.

In yet another embodiment, the amniotic fluid-derived cells of the present invention may be cryopreserved using commercially available medium containing DMSO (dimethylsulfoxide) or glycerol. The banked and frozen cells may be stored in the vapor phase of a liquid nitrogen storage tank until needed.

In yet another embodiment, the amniotic fluid-derived cells of the present invention may be transplanted with mature islets of the same or different animal species to enhance the survival of the amniotic fluid-derived cells or to induce further differentiation of the amniotic fluid-derived cells into a pancreatic β cell lineage.

The source of amniotic fluid from which the cells are isolated may be autologous in relation to the patient undergoing the therapeutic treatment. Alternatively, the source may be allogeneic, or xenogeneic. Cells to be administered to a patient may also be genetically modified to enhance proliferation and/or differentiation or prevent or lessen the risk of immune rejection. Alternatively, the amniotic fluid-derived cells obtained in accordance with the present invention can be used to modulate the recipient's immune response, prior to transplantation of differentiated cells prepared in accordance with the present invention. See, for example, U.S. Pat. No. 6,328,960, U.S. Pat. No. 6,281,012.

The amniotic fluid-derived cells of the present invention may be differentiated into an insulin-producing cell prior to transplantation into a recipient. In a specific embodiment, the amniotic fluid-derived cells of the present invention are fully differentiated into β-cells, prior to transplantation into a recipient. Alternatively, the amniotic fluid-derived cells of the present invention may be transplanted into a recipient in an undifferentiated or partially differentiated state. Further differentiation may take place in the recipient.

The amniotic fluid-derived cells of the present invention may be genetically modified. For example, the cells may be engineered to over express markers characteristic of a cell of a β-cell lineage, such as, for example, PDX-1or insulin. The cells may be engineered to over express with any suitable gene of interest. Furthermore, the cells may be engineered to over express markers characteristic of an intestinal cell, such as MATH-1. Alternatively, the cells of the present invention can be differentiated into a GIP expressing cell population and further modified with an insulin gene under control of the GIP promoter to become glucose responsive and insulin-producing cell population.

Techniques useful to genetically modify the amniotic fluid-derived cells of the present invention can be found, for example, in standard textbooks and reviews in cell biology. Methods in molecular genetics and genetic engineering are described, for example, in Molecular Cloning: A Laboratory Manual, 2nd Ed. (Sambrook et al., 1989); Oligonucleotide Synthesis (M. J. Gait, ed., 1984); Animal Cell Culture (R. I. Freshney, ed., 1987); the series Methods in Enzymology (Academic Press, Inc.); Gene Transfer Vectors for Mammalian Cells (I. M. Miller & M. P. Calos, eds. , 1987); Current Protocols in Molecular Biology and Short Protocols in Molecular Biology, 3rd Edition (F. M. Ausubel et al., eds., 1987 & 1995); and Recombinant DNA Methodology II (R. Wu ed., Academic Press 1995).

The nucleic acid molecule, encoding the gene of interest may be stably integrated into the genome of the host amniotic fluid-derived cell, or the nucleic acid molecule may be present as an extrachromosomal molecule, such as a vector or plasmid. Such an extrachromosomal molecule may be auto-replicating. The term “transfection,” as used herein, refers to a process for introducing heterologous nucleic acid into the host amniotic fluid-derived cell.

The cells, undifferentiated or otherwise, may be used as dispersed cells or formed into clusters that may be infused into the hepatic portal vein. Alternatively, the cells may be provided in biocompatible degradable polymeric supports, porous non-degradable devices or encapsulated to protect from host immune response. The cells may be implanted into an appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of implanted cells, additional factors, such as growth factors, antioxidants or anti-inflammatory agents, can be administered before, simultaneously with, or after the administration of the cells. In certain embodiments, growth factors are utilized to differentiate the administered cells in vivo. These factors can be secreted by endogenous cells and exposed to the administered amniotic fluid-derived cells in situ. Implanted amniotic fluid-derived cells can be induced to differentiate by any combination of endogenous and exogenously administered growth factors known in the art.

The amount of cells used in implantation depends on a number of factors including the patient's condition and response to the therapy, and can be determined by one skilled in the art.

In one aspect, this invention provides a method for treating a patient suffering from, or at risk of developing diabetes. The method includes isolating and culturing amniotic fluid-derived cells according to the present invention, expanding the isolated population of cells, differentiating in vitro the cultured amniotic fluid-derived cells into a β-cell lineage, and incorporating the cells into a three-dimensional support. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Support materials suitable for use for purposes of the present invention include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods of the present invention. See, e.g., the materials disclosed in U.S. Pat. No. 5,770,417, U.S. Pat. No. 6,022,743, U.S. Pat. No. 5,567,612, U.S. Pat. No. 5,759,830, U.S. Pat. No. 6,626,950, U.S. Pat. No. 6,534,084, U.S. Pat. No. 6,306,424, U.S. Pat. No. 6,365,149, U.S. Pat. No. 6,599,323, U.S. Pat. No. 6,656,488, and U.S. Pat. No. 6,333,029. Exemplary polymers suitable for use in the present invention are disclosed in U.S. Published Application 2004/0062753 A1 and U.S. Pat. No. 4,557,264.

To form a support incorporated with a pharmaceutical agent, the pharmaceutical agent can be mixed with the polymer solution prior to forming the support. Alternatively, a pharmaceutical agent could be coated onto a fabricated support, preferably in the presence of a pharmaceutical carrier. The pharmaceutical agent may be present as a liquid, a finely divided solid, or any other appropriate physical form. Alternatively, excipients may be added to the support to alter the release rate of the pharmaceutical agent. In an alternate embodiment, the support is incorporated with at least one pharmaceutical compound that is an anti-inflammatory compound, such as, for example compounds disclosed in U.S. Pat. No. 6,509,369.

In one embodiment, the support is incorporated with at least one pharmaceutical compound that is an anti-apoptotic compound, such as, for example, compounds disclosed in U.S. Pat. No. 6,793,945.

In another embodiment, the support is incorporated with at least one pharmaceutical compound that is an inhibitor of fibrosis, such as, for example, compounds disclosed in U.S. Pat. No. 6,331,298.

In a further embodiment, the support is incorporated with at least one pharmaceutical compound that is capable of enhancing angiogenesis, such as, for example, compounds disclosed in U.S. Published Application 2004/0220393 and U.S. Published Application 2004/0209901.

In still another embodiment, the support is incorporated with at least one pharmaceutical compound that is an immunosuppressive compound, such as, for example, compounds disclosed in U.S. Published Application 2004/0171623.

In a further embodiment, the support is incorporated with at least one pharmaceutical compound that is a growth factor, such as, for example, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-I (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, MAPK inhibitors, such as, for example, compounds disclosed in U.S. Published Application 2004/0209901 and U.S. Published Application 2004/0132729.

The incorporation of the cells of the present invention into a scaffold can be achieved by the simple depositing of cells onto the scaffold. Cells can enter into the scaffold by simple diffusion (J. Pediatr. Surg. 23 (1 Pt 2): 3-9 (1988)). Several other approaches have been developed to enhance the efficiency of cell seeding. For example, spinner flasks have been used in seeding of chondrocytes onto polyglycolic acid scaffolds (Biotechnol. Prog. 14(2): 193-202 (1998)). Another approach for seeding cells is the use of centrifugation, which yields minimum stress to the seeded cells and enhances seeding efficiency. For example, Yang et al. developed a cell seeding method (J. Biomed. Mater. Res. 55(3): 379-86 (2001)), referred to as Centrifugational Cell Immobilization (CCI).

The present invention is further illustrated, but not limited by, the following examples.

### EXAMPLE 1

### The Establishment of Human Amniotic Fluid-Derived Cell Lines

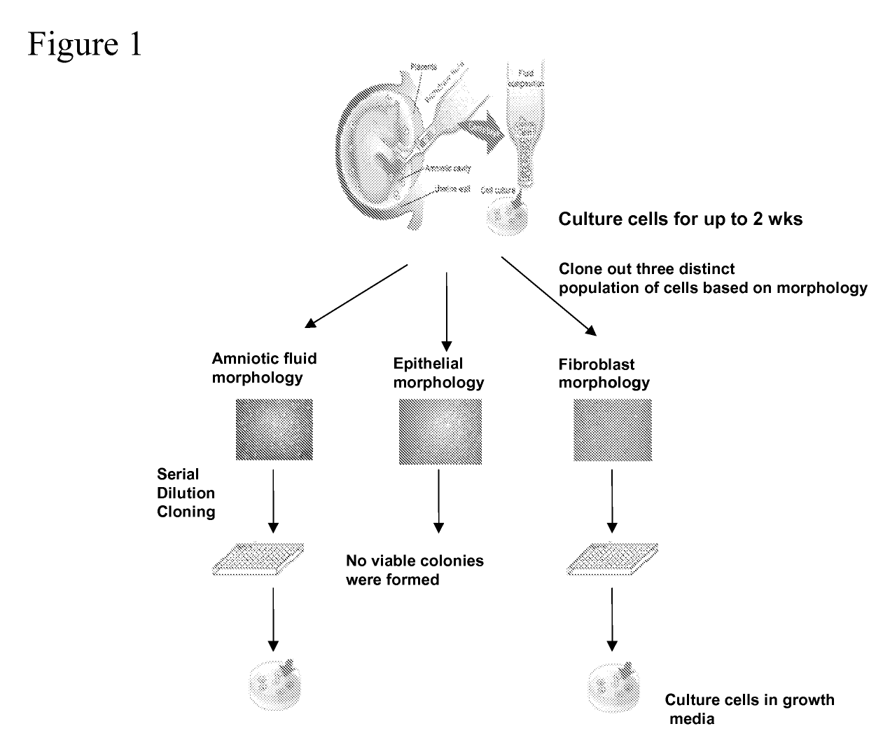

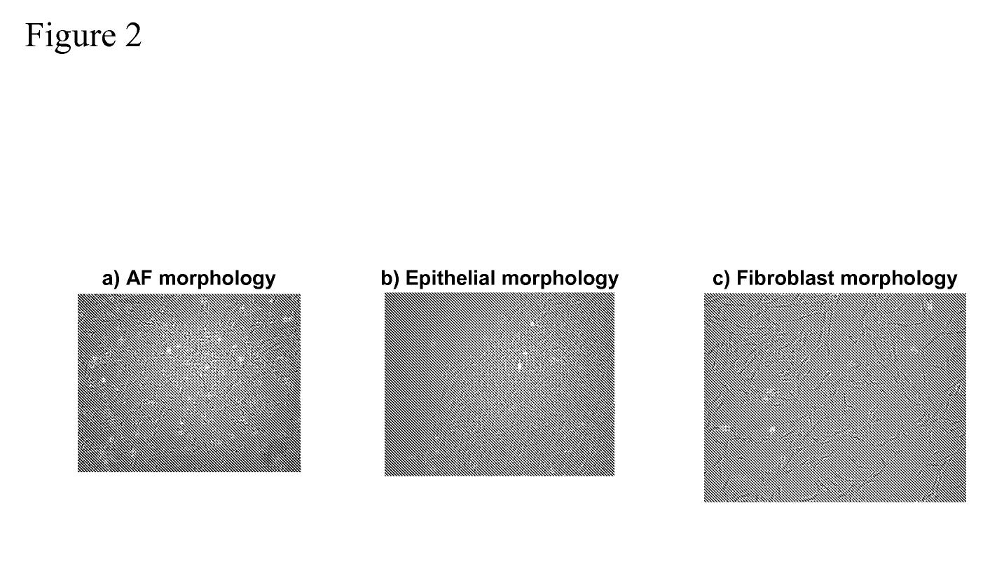

Amniotic fluid used to isolate the cells of the present invention was taken from samples taken from routine amniocentesis performed at 16 to 22 weeks of gestation for fetal karyotyping. The multi-stage method used to isolate the amniotic fluid-derived cells is outlined in FIG. 1. The amniotic fluid was centrifuged for 7 minutes at 400×g and the supernatant removed. The resulting cell pellet was resuspended in the growth media indicated in Table III for the amniotic fluid samples used in the present invention. The cells were cultured either on collagen type IV (1 mg/100 mm plate), or on collagen type I (1 microgram/cm²), vitronectin (10 microgram/ml) or fibronectin (10 micrograms/ml) coated plates. The cell yield from amniotic fluid samples had a large variation (8000-300000 cell/sample) and some samples also contained a significant number of blood cell contamination. The cultures were left undisturbed for at least 5-10 days under hypoxic conditions (3% O2). In parallel, cultures were established under similar conditions in normoxic conditions. Next, the cultures were fed with the same growth media and cultured until the cultures reached 70-80% confluency. Cells at this stage were referred to as “P0”. In some cultures, colonies of cells were isolated by a cloning ring and sub cultured into a different culture plate. Distinct colonies were present with morphologies characteristic of fibroblast (F), amniotic fluid (AF), and epithelial (E) cells (FIG. 2). Cells were released from P0 culture by using TrypLE Express™ (Invitrogen) and seeded into fibronectin, vitronectin, or collagen type IV coated flaks/dishes/plates at various densities (50-10,000 cell/cm2). Some of the P0 cells were used for serial dilution cloning. The population doubling time of the fastest growing cells was approximately 24 hrs at early passages. The expanded cells cultured under various media conditions (Table II) were analyzed for cell surface markers (Table III). Cells were typically split at ˜70% confluency and reseeded at 100-10000 cells/cm². RNA was collected at various stages of cell growth and analyzed for embryonic and germ layer markers (Table V).

Amniotic fluid cells of the present invention were present at various gestational ages. Table VI lists the presence or absence of AF, E, and F morphologies in amniotic fluid samples obtained at 17 weeks to 41 weeks of gestation.

Amniotic fluid cells of the present invention were also obtained from amniotic fluid obtained at term (approximately 40 wks of gestation). Amniotic fluid samples were obtained from 38-40 wk deliveries and cultured according to the protocols outlined above. The resulting adherent cell populations displayed very similar characteristics to the cells isolated from 16-22 wks of gestation.

### EXAMPLE 2

### Clonal Expansion of the Cells of the Present Invention

Using methods described in Example 1, cells with AF-like morphologies were harvested from P0 cultures using cloning rings. Three distinct populations of cells exhibiting different expression of surface receptors, cytoskeletal proteins, and transcription factors were identified. For sake of clarity, these populations are referred to as AF-I, AF-II, and AF-III cells. Subsequent examples highlight the differences between AF-I,-II, and-III populations.

### EXAMPLE 3

### Fluorescence-Activated Cell Sorting (FACS) Analysis

Adhered cells were removed from culture plates by five-minute incubation with the TRYPLE™ express solution (Gibco, CA). Released cells were resuspended in DMEM supplemented with 10% FBS and recovered by centrifugation, followed by washing and resuspending the cells in a staining buffer consisting of 2% BSA, 0.05% sodium azide (Sigma, MO) in PBS. If appropriate, the cells were Fc-receptor blocked using a 0.1% γ-globulin (Sigma) solution for 15 min. Aliquots (approximately 10⁵ cells) were incubated with either phycoerythirin (PE) or allophycocyanin (APC) conjugated monoclonal antibodies (5 μl antibody per 10⁶ cells), as indicated in Table III-A, or with an unconjugated primary antibody. Controls included appropriate isotype matched antibodies, non-stained cells, and cells only stained with secondary conjugated antibody. All incubations with antibodies were performed for 30 mins at 4° C., after which the cells were washed with the staining buffer. Samples that were stained with unconjugated primary antibodies were incubated for additional 30 mins at 4° C. with secondary conjugated PE or -APC labeled antibodies. See Table III-B for a list of secondary antibodies used. Washed cells were pelleted and resuspended in the staining buffer and the cell surface molecules were identified by using a FACS Array (BD Biosciences) by collecting at least 10,000 events.

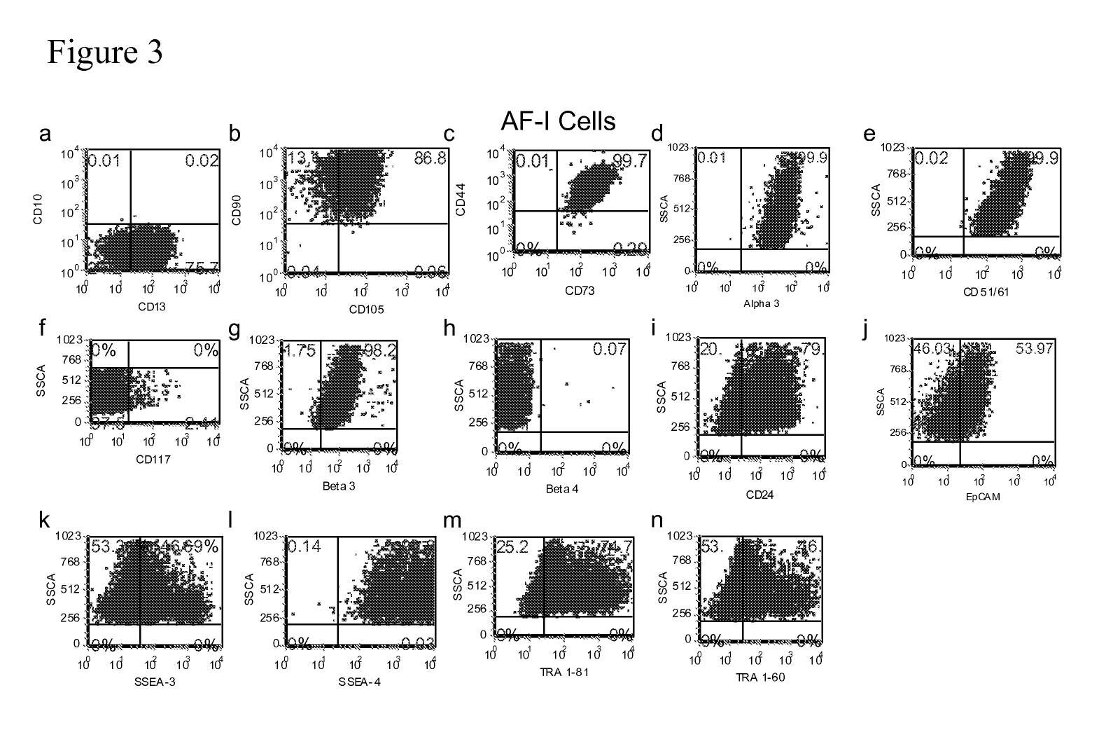

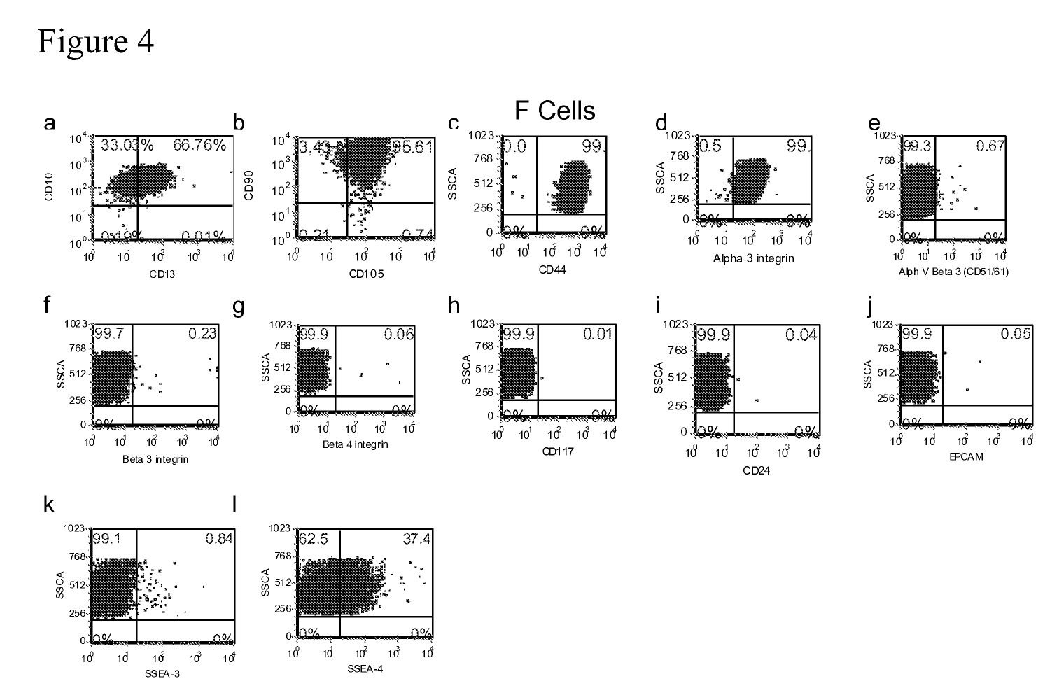

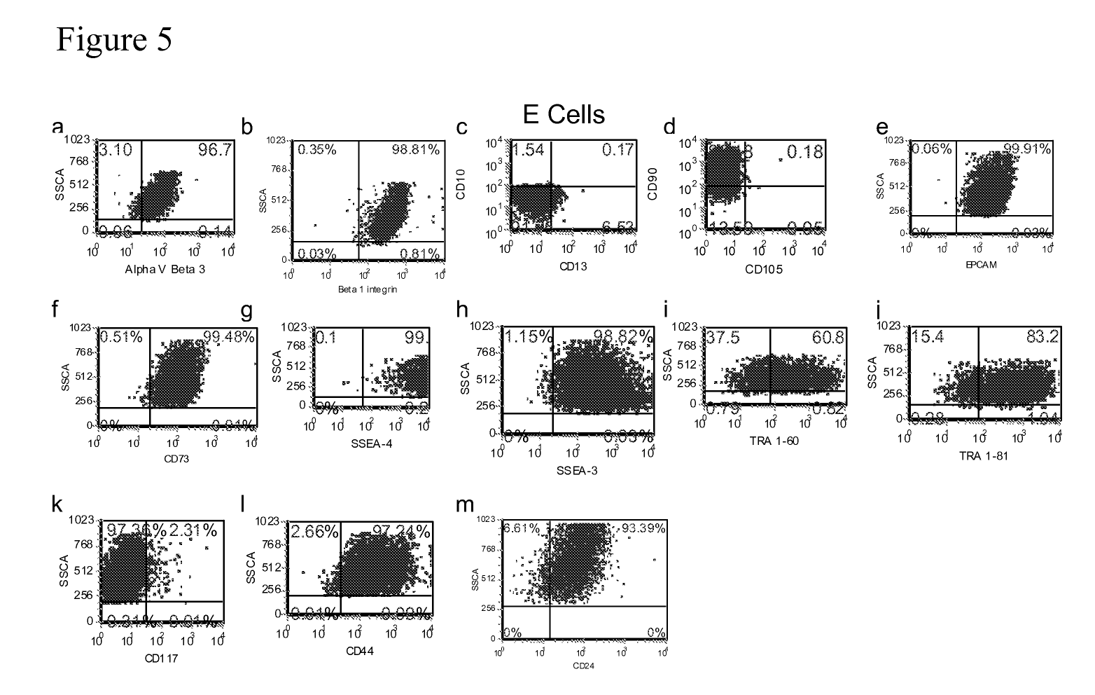

For intracellular staining, cells were first fixed for 10 mins with 4% paraformaldheyde, followed by two rinses in the staining buffer, centrifugation of cells and resuspension of the cells in a permeabilization buffer containing 0.5% Triton-X (Sigma) in PBS for 5 mins at room temperature (RT). The permeabilized cells were rinsed twice with a rinsing buffer, centrifuged, and resuspended in the staining buffer and incubated with an appropriate conjugated antibody (5 μl antibody per 10⁶ cells), for 30 mins at 4° C. Samples that were stained with unconjugated primary antibodies were incubated for additional 30 mins at 4° C. with secondary conjugated PE or -APC labeled antibodies (Table III B). Washed cells were pelleted and resuspended in the staining buffer and the internal proteins were identified by using a FACS Array (BD Biosciences) by collecting at least 10,000 events. The expression level of examined surface and internal markers is listed in Table IV A and B. FACS analysis allowed identification of signature markers to distinguish amniotic fluid cells (AF-I, -II, and -III), fibroblasts (F), and epithelial cells (E) (FIGS. 3-5). Table IV C lists the cell surface expression profile of AF-I cells isolated from term (38-40 wks) amniotic fluid. The expression level of cell surface receptors is very similar to AF-I cells isolated from 16-22 wks amniotic fluid.

### EXAMPLE 4

### Immunostaining of Undifferentiated Cells

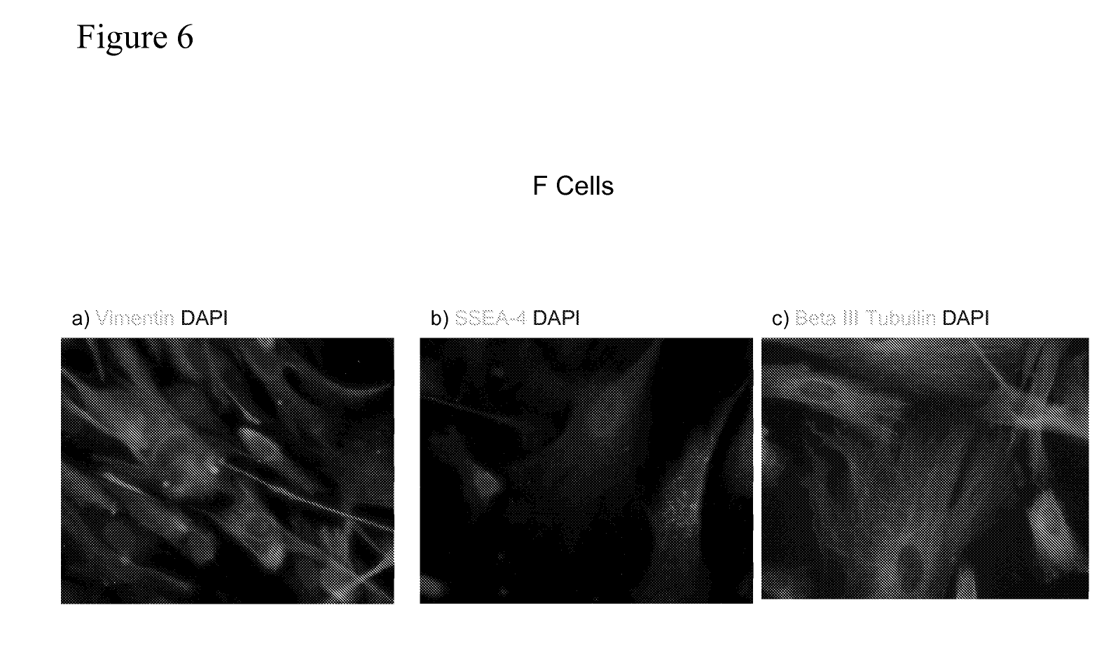

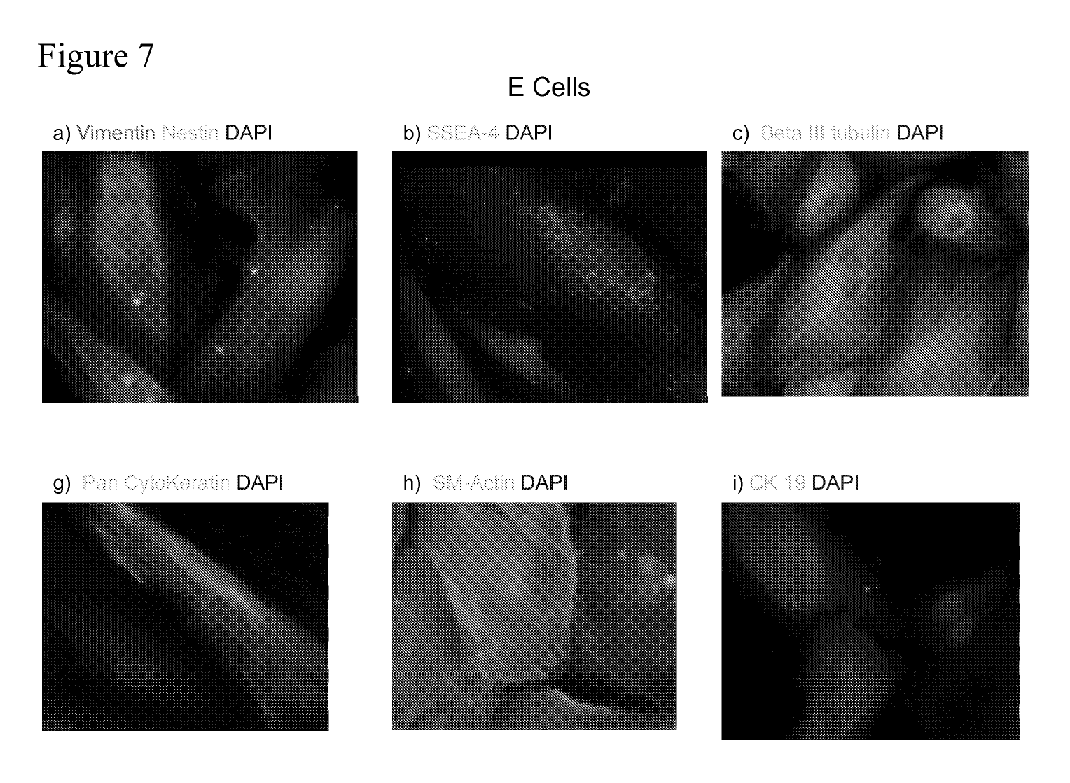

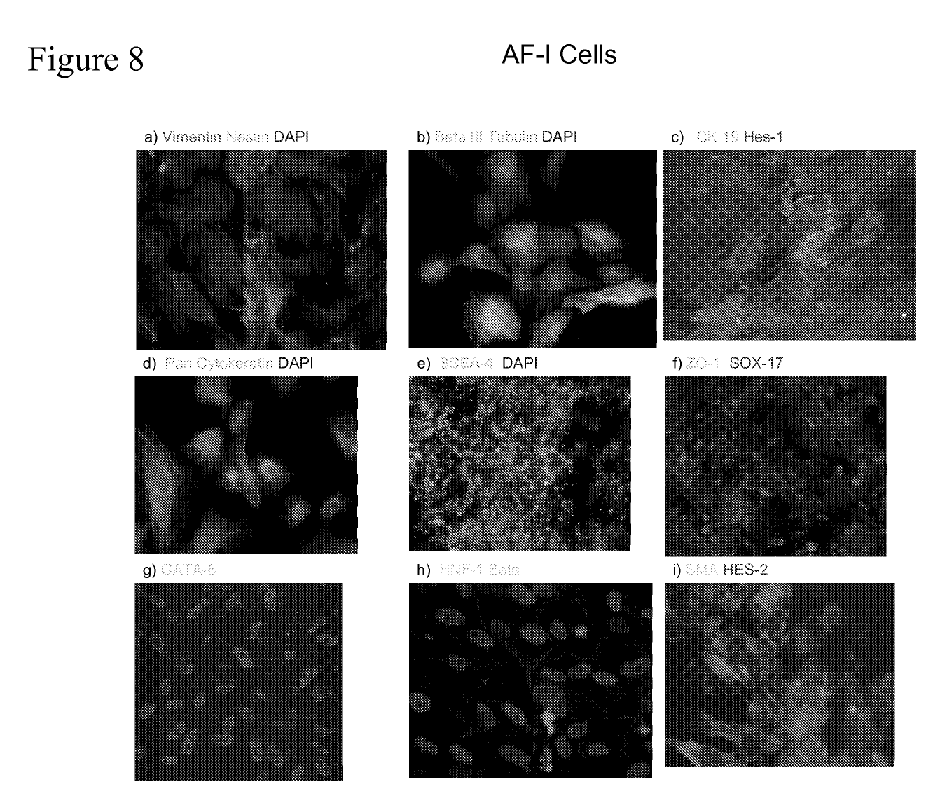

10,000 cells/cm² cells, cultured according to Example 1, were seeded into glass bottom 35 mm microwell dishes (Matek Corp, MA) in various growth media. Following three days in culture, the cells were fixed for 10 mins with 4% paraformaldheyde, followed by two rinses in the PBS, and addition of a permeabilization buffer containing 0.5% Triton-X (Sigma) for 5 mins at room temperature (RT) followed by additional three rinses with PBS. The fixed and permeabilized cells were blocked with either 1% bovine serum albumin (BSA) or 4% sera from the species where the secondary antibody was raised in (Goat, donkey, or rabbit). Control samples included reactions with the primary antibody omitted or where the primary antibody was replaced with corresponding immunoglobulins at the same concentration as the primary antibodies. Stained samples were rinsed with a PROLONG® antifade reagent (Invitrogen, CA) containing diamidino-2-phenylindole, dihydrochloride (DAPI) to counter stain the nucleus. Images were acquired using a Nikon Confocal Eclipse C-1 inverted microscope (Nikon, Japan) and a 10-60× objective (FIGS. 6-8).

### EXAMPLE 5

### PCR Analysis of Undifferentiated Cells

RNA was extracted from cells cultured in the growth media. Total RNA from human pancreas, liver, brain, gut (Ambion, INC.) NTERA cells (human embryonic carcinoma cells line, ATCC), HEK293 cells (ATCC), and human airway epithelia cells (Cambrex) were used as positive controls. Bone marrow derived mesenchymal cells (Cambrex, MD) were used as negative controls for the expression of key genes involved in pancreatic development.

RNA extraction, purification, and CDNA synthesis. RNA samples were purified through its binding to a silica-gel membrane (Rneasy Mini Kit, Qiagen, CA) in the presence of an ethanol-containing, high-salt buffer; while contaminants were washed away. The RNA was further purified while bound to the column by treatment with DNase I (Qiagen, CA) for 15 min. High-quality RNA was then eluted in water. Yield and purity were assessed by A260 and A280 readings on the spectrophotometer. cDNA copies were made from purified RNA using an ABI (ABI, CA) high capacity cDNA archive kit.

Real-time PCR amplification and quantitative analysis. Unless otherwise stated, all reagents were purchased from Applied Biosystems. Real-time PCR reactions were performed using the ABI PRISM® 7000 Sequence Detection System. TAQMAN® UNIVERSAL PCR MASTER MIX® (ABI, CA) was used with 20 ng of reverse transcribed RNA in a total reaction volume of 20 μl. Each cDNA sample was run in duplicate to correct for pipetting errors. Primers and FAM-labeled TAQMAN® probes were used at concentrations of 200 nM. The level of expression of each target gene was normalized using the pre-developed Applied Biosystem's 18S ribosomal RNA or human glyceraldehydes-3-phosphate dehydrogenase (GAPDH) endogenous control kit. Primers and probes were either designed using ABI PRISM PRIMER EXPRESS™ software or used pre-developed ABI gene analysis kit. For each gene, either one of the primers or the probe were designed to be exon-boundary spanning. This eliminated the possibility of the primers/probe binding to any genomic DNA present. The primer and probe sets are listed as following Nkx2.2 (Hs00159616), Pdx-1 (Hs00426216), Nkx6.1 (Hs00232355), Ngn3 (Hs00360700), Pax4 (Hs00173014), Pax6 (Hs00240871), Insulin (Hs00355773), Glu2 (Hs00165775), glucagon (Hs00174967), Isl-1 (Hs00158126), somatostatin (Hs00174949), FoxA2 (HNF 3-beta) (Hs00232764), HlxB9 (Hs00232128), GATA-4 (Hs00171403), HNF1β (Hs00172123), Musashi Homolog 1 (Msi-1) (Hs00159291), Hes-1 (Hs00172878), Neurotensin (NTS) (Hs00175048), Cholecystokinin (Hs00174937), AFP (Hs00173490), Secretin (Hs00360814), GIP (Hs00175030), GFAP (Hs00157674), MAP2 (Hs00159041), Olig2 (Hs0037782), Oct-4 (CGACCATCTGCCGCTTTGAG (SEQ ID NO: 1) and CCCCCTGTCCCCCA TTCCTA (SEQ ID NO: 2)); Rex-1 (CAGATCCTAAACAGCTCGCAGAAT (SEQ ID NO: 3), and GCGTACGCAAATTAAACTCCAGA(SEQ ID NO: 4); Sox17: TGGCGCAGCAGATACCA (SEQ ID NO:5), AGCGCCTTCCACGACTTG (SEQ ID NO:6) and CCAGCATCTTGCTCAACTCGGCG (SEQ ID NO:7); ABCG-2: GTTTATCCGTGGTGTGTCTGG (SEQ ID NO: 8) and CTGAGCTATAGAGGCCTGGG (SEQ ID NO: 9); SOX2: ATGCACCGCTACGACGTGA (SEQ ID NO:10) and CTTTTGCACCCCTCCCATTT (SEQ ID NO: 11). The remaining primers were designed by using the PRIMERS program (ABI, CA) and are listed in Table V. After an initial 50° C. for 2 min, and 95° C. for 10 min, samples were cycled 40 times in two stages-a denaturation step at 95° C. for 15 sec, followed by an annealing/extension step at 60° C. for 1 min. Data analysis was carried out using GENEAMP®7000 Sequence Detection System software. For each primer/probe set, a Cₜ value was determined as the cycle number at which the fluorescence intensity reached a specific value in the middle of the exponential region of amplification. Relative gene expression levels were calculated using the comparative Cₜ method. Briefly, for each cDNA sample, the endogenous control Cₜ value was subtracted from the gene of interest Cₜ to give the delta Cₜ value (ΔCₜ). The normalized amount of target was calculated as 2^{-ΔCt}, assuming amplification to be 100% efficiency. Final data were expressed relative to a calibrator sample. The comparative Cₜ method is only valid if target and endogenous control amplification efficiencies are approximately equal. Preliminary validation experiments were therefore performed for each primer/probe set by amplifying serially diluted cDNA samples and determining the ΔCₜ values. These ΔCₜ values should remain constant across the range of dilutions if amplification efficiencies are equal (Table V).

### EXAMPLE 6

### Population Doubling Time

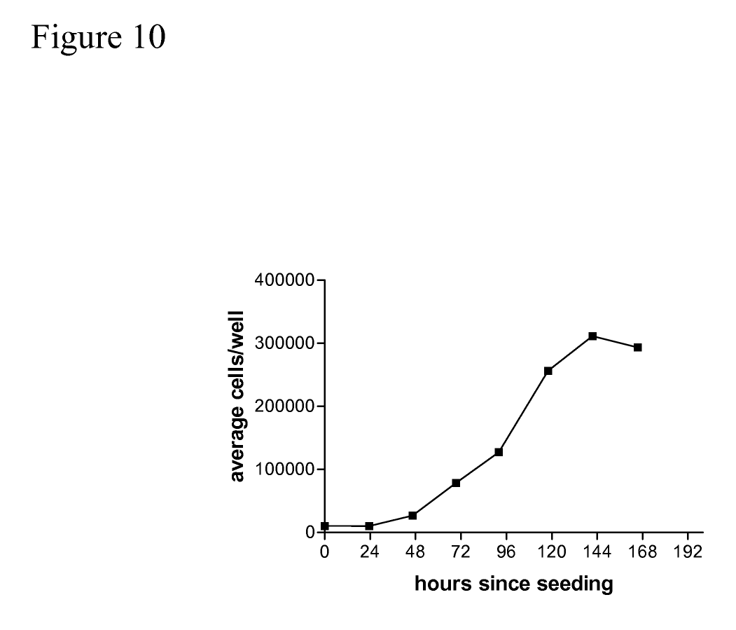

Passage 6 amniotic fluid cells (AF-I type), isolated and expanded according to Example 1 were seeded at 10000 cells/well of a 24-well tissue culture plate (Corning, MA) in growth media #11. At various time points, cells were removed from three wells of the plate using TRYPLE™ Express (Invitrogen, CA) and counted using a Guava PCA-96 cell analysis system and the VIACOUNT® reagent (Guava, CA). FIG. 10 depicts the growth curve of passage 6 cells cultured under hypoxic conditions (3% O2). The linear phase of the log plot was used to estimate the population doubling time of the cells. Population doubling time of passage 6 cells was 31 hrs.

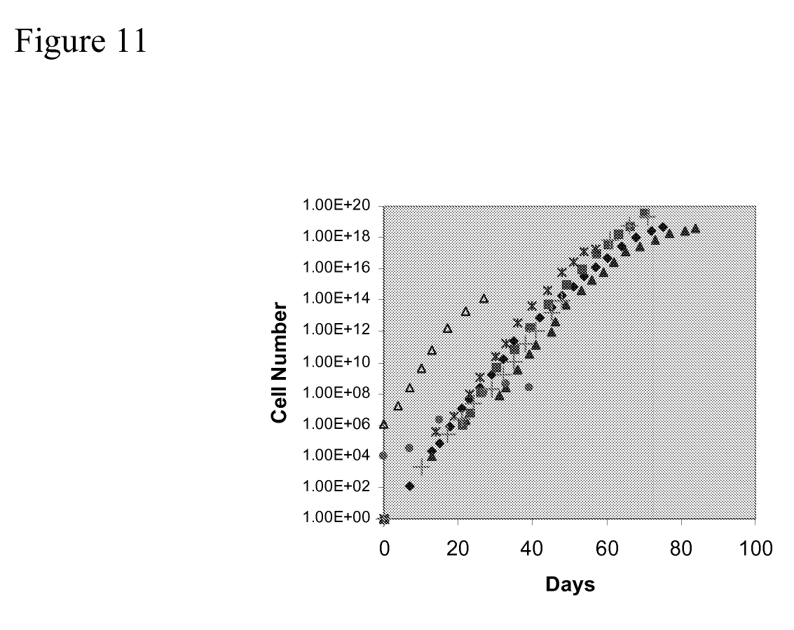

The growth potential of the three cell populations (fibroblast, AF, and epithelial morphology) were compared over long-term cultures. FIG. 11 depicts the growth potential of AF-I, AF-II, AF-III, F, and E cells cultured in media #5. It is clear that F (“fibroblastic” amniotic fluid-derived) cells and AF cells can expand well above 50 population doublings and represent a scalable source for cell therapy applications.

### EXAMPLE 7

### Telomere Length of AF-I Cells

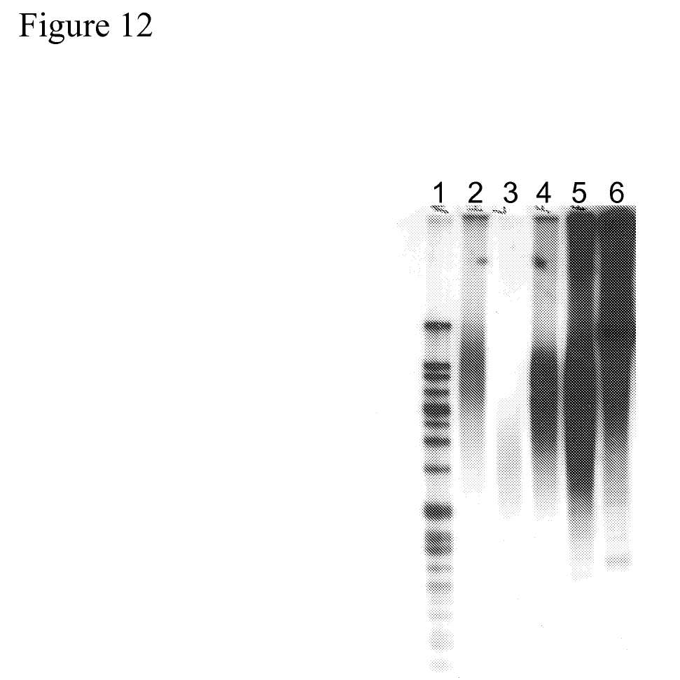

The telomere length of an AF-I line isolated from a single cell by limited serial dilution was analyzed at passage 12 (approximately 50 population doublings) by using the Telo TAGGG Telomere Length Assay (Roche, IN) and following the manufacturer's instruction. The telomere length was analyzed for cells cultured in DMEM-LG+10% FBS and cells cultured in Amniomax™ (Gibco) see FIG. 12. DNA from NTERA cells served as a positive control.

### EXAMPLE 8

### Karyotype Analysis

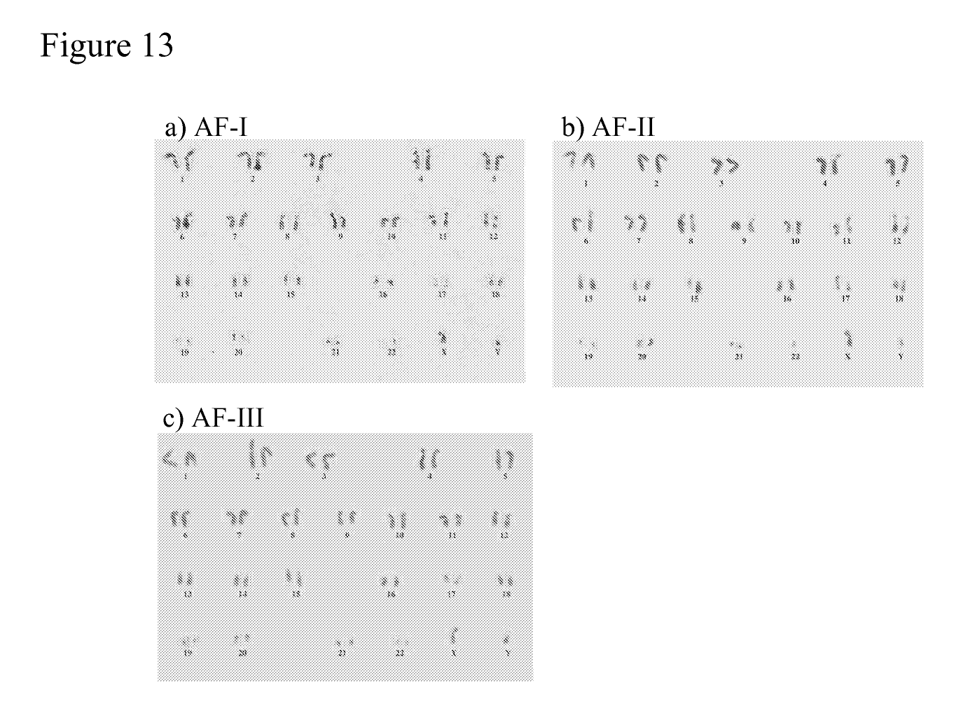

The karyotype of AF cells, isolated from mutliple donors at passage 8-10 (approximately 30 population doublings), was determined by G-band analysis. Five karyotypes were prepared and cytogenetic analysis showed that the cells had a normal autosomes and a modal chromosome number of 46. All cells analyzed also contained the X and Y-chromosomes confirming their fetal origin. FIG. 13 depicts karyotypes of amniotic fluid-derived cells (AF-I, AF-II, and AF-III) isolated from amniotic fluid obtained from 16-22 weeks of gestation.

### EXAMPLE 9

### Expansion Potential of AF Cells Derived from Term Amniotic Fluid

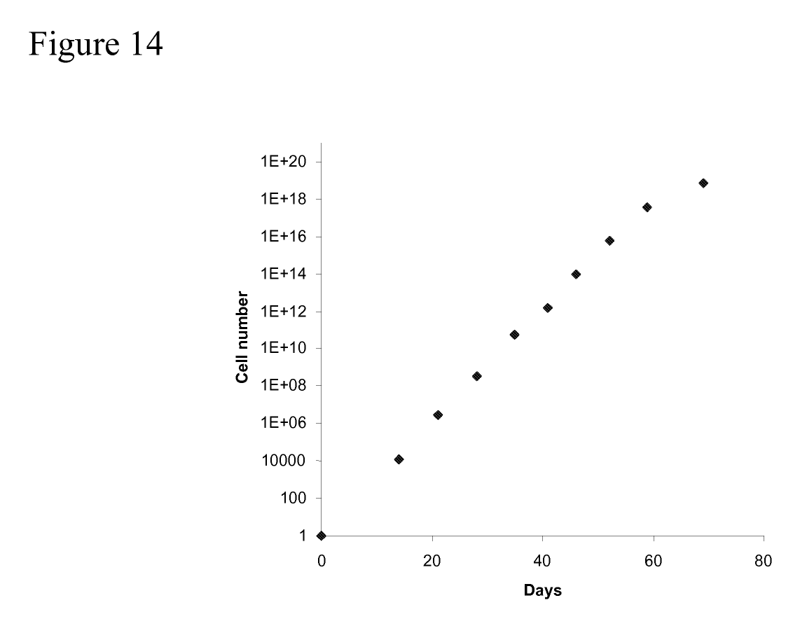

FIG. 14 depicts the expansion potential of an AF-I cell morphology derived from term amniotic fluid (˜38 weeks) and cultured in media #5. The expansion potential is very similar to the AF-I cells isolated from 16-22 wks amniotic fluid.

### EXAMPLE 10

### Microarray Analysis of Fibroblast, Epithelial, and Amniotic Fluid Morphology Cells

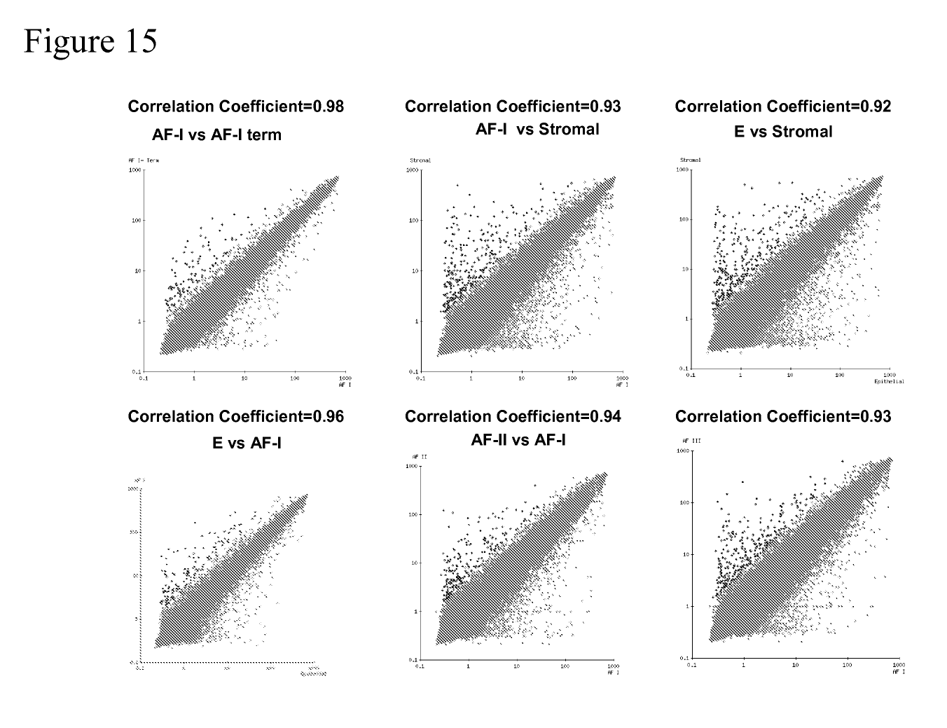

Total RNA was isolated from passage 9-11 amniotic fluid-derived fibroblast cells (F), amniotic fluid-derived epithelial cells (E), amniotic fluid-derived amniotic fluid cells (AF-I, -II, and -III lines), and amniotic fluid at term (AF term) using an RNeasy mini kit (Qiagen). The sample preparation, hybridization, and image analysis was performed according to the CodeLinkTM System (GE Healthcare, Amersham Biosciences, NJ). CodelinkTm Human Whole Genome arrays were used. It is comprised of approximately 55 000 30-mer probes designed to conserved exons across the transcripts of targeted genes. The chip contains ˜45000 unique Unigene IDs. Following normalization and a log transformation, data analysis was performed using OmniViz® software (MA) and GENESIFTER (VizXLabs, WA). The variance stabilizing transformation along with cross sample normalization was applied to the log transformed array dataset. The variability within each cell line and among the different cell lines was compared using the Pearson correlation coefficient. For all the samples analyzed, the correlation coefficient within a cell line was higher as compared to those between the lines. Variance in gene expression profiles between the different cell types along with the correlation coefficient between the lines are depicted in FIG. 15. Significant differences in gene expression between the cell types were evaluated using analysis of variance and an F-test with adjusted P-value (Benjamini and Hochberg correction) of ≦0.05. Tables VII A-G list the genes that are differentially expressed at least 5-fold between the various cell types.

### EXAMPLE 11

### Differentiation of Cells into Intestinal-Like Cells

The AF-I line, AFCA007 Clone A passage 14, was cultured at 10,000 cells/cm² in AMNIOMAX™. At confluency, the cells were further treated for 2 weeks with a daily dose of 10 micro molar retinoic acid (RA) in AMNIOMAX™ media. RNA was collected at day 14 and expression of intestinal hormones (secretin, neurotensin, gastric inhibitory peptide (GIP), cholecystokinin, somatostatin, and gastrin) was assessed by using real-time PCR as outlined in Example 4. Intestinal RNA (Ambion) was used to assess relative levels of expression using the ΔΔCₜ method. Table VIII lists the Cₜ values and the relative level of expression of the intestinal hormones in treated and untreated samples. As shown in Table VIII, addition of RA enhanced expression of the gut hormones.

### EXAMPLE 12

### Differentiation of Cells into Multiple Endocrine Lineages

Cells from the cell line AFCA007 Clone A (AF-I) at passage 8 were embedded in collagen type I (Becton Dickinson, CA) with 1% growth-factor reduced matrigel matrix (Becton Dickinson), and seeded into 6-well transwell insert at 5×10⁵ cells per well. The bottom well was seeded with human aortic endothelial cells passage 6 (Cambrex. MD). Cells were cultured with DMEM medium supplemented with 5% FBS and growth factors, which includes Cyclopamine, bFGF, EGF, BMP4-7, Activin A, Exendin 4, FGF4, all-trans retinoic acid and γ-secretase inhibitors for 14 days. Cultures were fed every other day. Cells treated by all-trans retinoic acid showed the up-regulation of alpha-fetoprotein (AFP). Treatment of cells with Activin A, BMP4, or the γ-secretase inhibitor L-685,458 up-regulated the expression of HNF-3 beta. Treatment of cells with BMPs at high concentration, 50 ng/ml, also up-regualted the GATA4 expression. Treatment of cells with FGF4 at 50 ng/ml showed an up-regulation of PDX-1expression (Table IX).

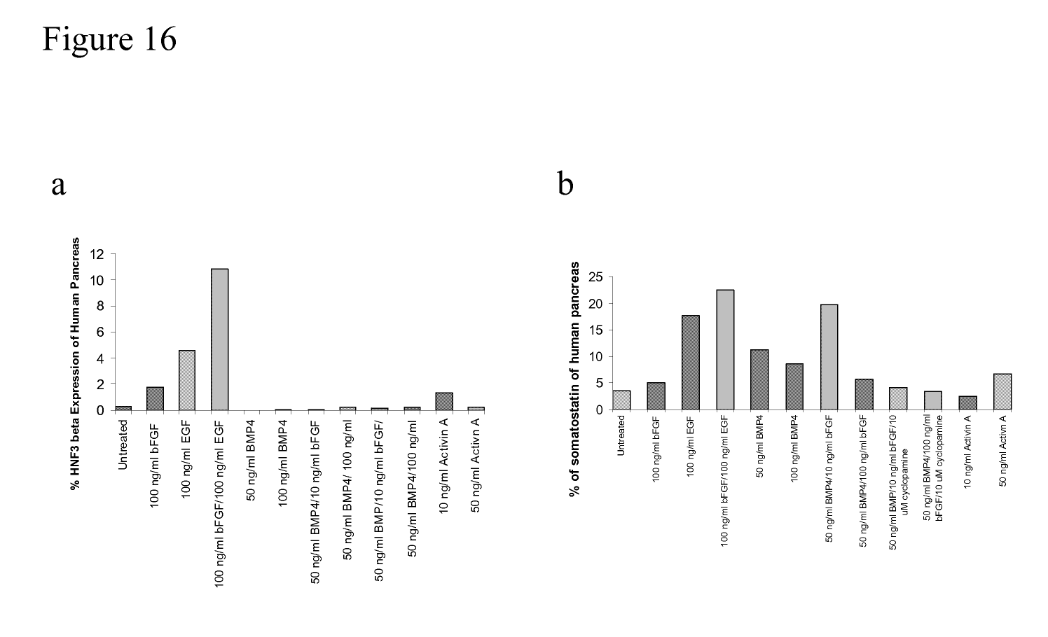

Cells from the cell line AFCA004 (E morphology) at passage 6 were seeded at 5×10⁵ cells per well of 6-well culture plates and treated with conditioned medium from confluent PANC-1 cells (ATCC, VA) in combination with different growth factors. Basic FGF, EGF and combination of bFGF and EGF enhanced the expression of HNF-3 beta ˜100 fold over untreated cells. Basic FGF, EGF and BMPs also stimulated somatostatin expression after 14 days treatment (FIG. 16, panels a&b).

Taken together, these results suggest that AF cells could be differentiated into pancreatic, hepatic or intestinal lineage by treating the cells with different growth factors.

### EXAMPLE 13

### Modulation of the Expression of Endoderm Makers by Inhibiting the Notch Pathway

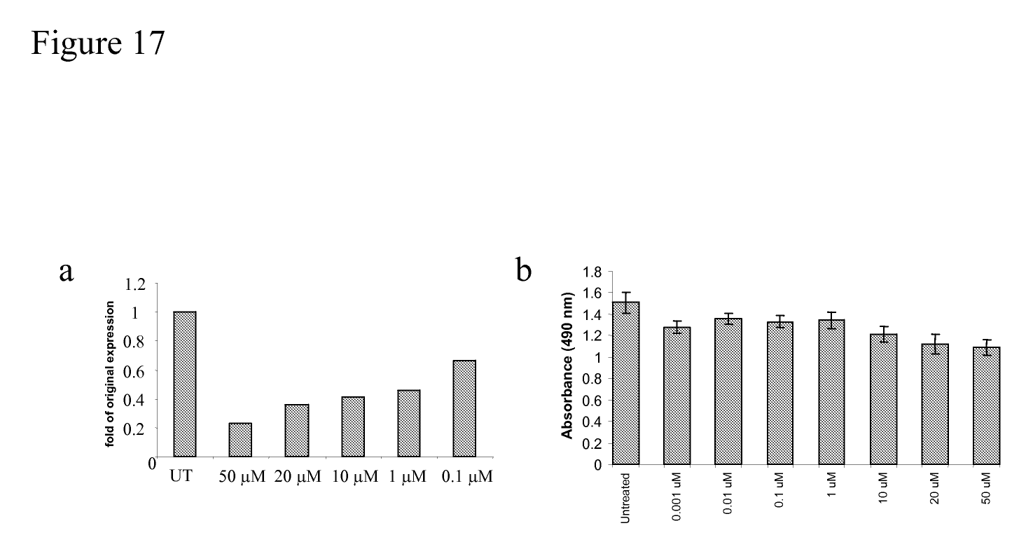

Cells from the cell line AFCA007 (AF-I) at passage 8 were treated with a range of concentrations of the notch pathway inhibitor L-685,458 (Sigma, MO) for 3 to 5 days. The cytotoxcity of L-685,458 was determined by measuring cell viability by a MTS assay (Promega, WI). Real-time PCR analysis was performed to analyze Hes-1 expression after the treatment. We found that L-685,458 showed a dose-dependent inhibitory effect on Hes-1 expression, Hes-1 is the downstream direct target of Notch pathway (FIG. 17, panel a). No effect on cell viability, as determined by the MTS assay, was observed following L-685,458 treatment of up to 10 μM for 5 days (FIG. 17, panel b).

### EXAMPLE 14

### Cytokine Antibody Array Analysis for AF I and AF II Cells

AFCA007 A (AF-I) and AFCA015 C (AF-II) at passage 10 were grown to approximately 70% confluency and then cell lystaes was collected using mammalian cell lysis kit (Sigma-Aldrich, MO). Cytokine array analyss was completed using Cytokine Array panels provided by RayBiotech, GA (http://www.raybiotech.com/). Table X lists cytokine, cytokine and growth factor receeptor expression following normalization of the data and background subtraction. For each panel, positive and negative controls are also included. The panels were run for two different samples per cell type.

LENGTHY TABLE REFERENCED HERE
US20070122903A1-20070531-T00001
Please refer to the end of the specification for access instructions.

LENGTHY TABLE REFERENCED HERE
US20070122903A1-20070531-T00002
Please refer to the end of the specification for access instructions.

LENGTHY TABLE REFERENCED HERE
US20070122903A1-20070531-T00003
Please refer to the end of the specification for access instructions.

LENGTHY TABLE REFERENCED HERE
US20070122903A1-20070531-T00004
Please refer to the end of the specification for access instructions.

LENGTHY TABLE REFERENCED HERE
US20070122903A1-20070531-T00005
Please refer to the end of the specification for access instructions.

LENGTHY TABLE REFERENCED HERE
US20070122903A1-20070531-T00006
Please refer to the end of the specification for access instructions.

LENGTHY TABLE REFERENCED HERE
US20070122903A1-20070531-T00007
Please refer to the end of the specification for access instructions.

LENGTHY TABLE REFERENCED HERE
US20070122903A1-20070531-T00008
Please refer to the end of the specification for access instructions.

LENGTHY TABLE REFERENCED HERE
US20070122903A1-20070531-T00009
Please refer to the end of the specification for access instructions.

LENGTHY TABLE REFERENCED HERE
US20070122903A1-20070531-T00010
Please refer to the end of the specification for access instructions.

LENGTHY TABLE REFERENCED HERE
US20070122903A1-20070531-T00011
Please refer to the end of the specification for access instructions.

LENGTHY TABLE REFERENCED HERE
US20070122903A1-20070531-T00012
Please refer to the end of the specification for access instructions.

LENGTHY TABLE REFERENCED HERE
US20070122903A1-20070531-T00013
Please refer to the end of the specification for access instructions.

LENGTHY TABLE REFERENCED HERE
US20070122903A1-20070531-T00014
Please refer to the end of the specification for access instructions.

LENGTHY TABLE REFERENCED HERE
US20070122903A1-20070531-T00015
Please refer to the end of the specification for access instructions.

LENGTHY TABLE REFERENCED HERE
US20070122903A1-20070531-T00016
Please refer to the end of the specification for access instructions.

LENGTHY TABLE REFERENCED HERE
US20070122903A1-20070531-T00017
Please refer to the end of the specification for access instructions.

LENGTHY TABLE REFERENCED HERE
US20070122903A1-20070531-T00018
Please refer to the end of the specification for access instructions.

LENGTHY TABLE REFERENCED HERE
US20070122903A1-20070531-T00019
Please refer to the end of the specification for access instructions.

LENGTHY TABLE REFERENCED HERE
US20070122903A1-20070531-T00020
Please refer to the end of the specification for access instructions.

LENGTHY TABLE REFERENCED HERE
US20070122903A1-20070531-T00021
Please refer to the end of the specification for access instructions.

Publications cited throughout this document are hereby incorporated by reference in their entirety. Although the various aspects of the invention have been illustrated above by reference to examples and preferred embodiments, it will be appreciated that the scope of the invention is defined not by the foregoing description, but by the following claims properly construed under principles of patent law.

LENGTHY TABLE
The patent application contains a lengthy table section. A copy of the table is available in electronic form from the USPTO web site (http://seqdata.uspto.gov/?pageRequest=docDetail&DocID=US20070122903A1). An electronic copy of the table will also be available from the USPTO upon request and payment of the fee set forth in 37 CFR 1.19(b)(3).

What is claimed is:

1. A substantially pure population of amniotic fluid-derived cells.

2. The population of amniotic fluid-derived cells according to claim 1, wherein the cells of said population are substantially negative in the expression of at least one protein marker selected from the group consisting of: CD117, Oct4, and Tra2-54.

3. The population of amniotic fluid-derived cells according to claim 2, wherein the cells of said population are substantially positive for the expression of at least one protein marker selected from the group consisting of: HNF-1 beta, GATA-6, and Sox-17.

4. The population of amniotic fluid-derived cells according to claim 2, wherein the cells of said population also express at least one gene selected from the group consisting of: HNF-3 beta, Hes-1, GATA-4 and Musashi-1.

5. The population of amniotic fluid-derived cells according to claim 2, wherein the cells of said population are positive for the expression of at least one protein marker selected from the group consisting of: Liver Activator Protein, PDGF receptor β, AXL, bFGF, EGF receptor, Fas/TNFRSF6, GRO, GRO-alpha, ICAM-1, IL-1 alpha, Il-3, Il-6, Il-8, MIF, Osteoprotegerin, TIMP-2, and TRAIL R3.

6. The population of amniotic fluid-derived cells according to claim 2, wherein the cells of said population are substantially negative in the expression of the protein marker Sox-17.

7. The population of amniotic fluid-derived cells according to claim 6, wherein the cells of said population are substantially negative in the expression of cytokeratin protein.

8. The population of amniotic fluid-derived cells according to claim 6, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

9. The population of amniotic fluid-derived cells according to claim 1, wherein the cells of said population are substantially negative in the expression of the protein marker Sox-17, and also express at least one gene selected from the group consisting of: Hes-1 and Musashi-1.

10. The population of amniotic fluid-derived cells according to claim 1, wherein the cells of said population are substantially negative in the expression of CD 117, Oct4, Sox-17, and Tra2-54.

11. The population of amniotic fluid-derived cells according to claim 10, wherein the cells of said population are substantially negative in the expression of cytokeratin protein.

12. The population of amniotic fluid-derived cells according to claim 10, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

13. The population of amniotic fluid-derived cells according to claim 10, wherein the cells of said population are substantially negative in the expression of the protein marker Sox-17, and also express at least one gene selected from the group consisting of: Hes-1 and Musashi-1.

14. The population of pancreatic amniotic fluid-derived cells according to claim 1, capable of propagating in vitro.

15. The population of amniotic fluid-derived cells according to claim 1, capable of propagating in vitro under hypoxic conditions.

16. The population of amniotic fluid-derived cells according to claim 1, capable of differentiating into cells displaying the characteristics of the β-cell lineage.

17. The population of amniotic fluid-derived cells according to claim 1, capable of differentiating into a gut hormone-producing cell.

18. A method of obtaining a population of cells from amniotic fluid, comprising:
a. Isolating amniotic fluid,
b. Isolating the cells from the amniotic fluid,
c. Placing the cells in culture medium,
d. Plating the cells in a culture vessel, and
e. Allowing the cells to grow in said medium for at least about five days, thereby obtaining a population of amniotic fluid-derived cells.

19. The method according to claim 18, wherein the cells of said population are substantially negative in the expression of at least one protein marker selected from the group consisting of: CD117, Oct4, and Tra2-54.

20. The method according to claim 19, wherein the cells of said population are substantially positive for the expression of at least one protein marker selected from the group consisting of: HNF-1 beta, GATA-6, and Sox-17.

21. The method according to claim 19, wherein the cells of said population also express at least one gene selected from the group consisting of: HNF-3 beta, Hes-1, GATA-4 and Musashi-1.

22. The method according to claim 19, wherein the cells of said population are positive for the expression of at least one protein marker selected from the group consisting of: Liver Activator Protein, PDGF receptor β, AXL, bFGF, EGF receptor, Fas/TNFRSF6, GRO, GRO-alpha, ICAM-1, IL-1 alpha, Il-3, Il-6, Il-8, MIF, Osteoprotegerin, TIMP-2, and TRAIL R3.

23. The method according to claim 19, wherein the cells of said population are substantially negative in the expression of the protein marker Sox-17.

24. The method according to claim 23, wherein the cells of said population are substantially negative in the expression of cytokeratin protein.

25. The method according to claim 23, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

26. The method according to claim 18, wherein the cells of said population are substantially negative in the expression of the protein marker Sox-17, and also express at least one gene selected from the group consisting of: Hes-1 and Musashi-1.

27. The method according to claim 18, wherein the cells of said population are substantially negative in the expression of CD117, Oct4, Sox-17, and Tra2-54.

28. The method according to claim 27, wherein the cells of said population are substantially negative in the expression of cytokeratin protein.

29. The method according to claim 27, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

30. The method according to claim 27, wherein the cells of said population are substantially negative in the expression of the protein marker Sox-17, and also express at least one gene selected from the group consisting of: Hes-1 and Musashi-1.

31. The method according to claim 18, wherein the cells of said population are capable of propagating in vitro.

32. The method according to claim 18, wherein the cells of said population are capable of propagating in vitro under hypoxic conditions.

33. The method according to claim 18, wherein the cells of said population are capable of differentiating into cells displaying the characteristics of the β-cell lineage.

34. The method according to claim 18, wherein the cells of said population are capable of differentiating into a gut hormone-producing cell.

35. A method of obtaining a population of cells from amniotic fluid, comprising:
a. Isolating amniotic fluid,
b. Isolating the cells from the amniotic fluid,
c. Selecting cells that express at least one of the markers selected from the group consisting of SSEA-4, SSEA-3, TRA1-60 and TRA1-81,
d. Placing the cells in culture medium,
e. Plating the cells in a culture vessel, and
f. Allowing the cells to grow in said medium for at least about five days, thereby obtaining a population of amniotic fluid-derived cells.

36. The method according to claim 35, wherein the cells of said population are substantially negative in the expression of at least one protein marker selected from the group consisting of: CD117, Oct4, and Tra2-54.

37. The method according to claim 36, wherein the cells of said population are substantially positive for the expression of at least one protein marker selected from the group consisting of: HNF-1 beta, GATA-6, and Sox-17.

38. The method according to claim 36, wherein the cells of said population also express at least one gene selected from the group consisting of: HNF-3 beta, Hes-1, GATA-4 and Musashi-1.

39. The method according to claim 36, wherein the cells of said population are positive for the expression of at least one protein marker selected from the group consisting of: Liver Activator Protein, PDGF receptor β, AXL, bFGF, EGF receptor, Fas/TNFRSF6, GRO, GRO-alpha, ICAM-1, IL-1 alpha, Il-3, Il-6, Il-8, MIF, Osteoprotegerin, TIMP-2, and TRAIL R3.

40. The method according to claim 36, wherein the cells of said population are substantially negative in the expression of the protein marker Sox-17.

41. The method according to claim 40, wherein the cells of said population are substantially negative in the expression of cytokeratin protein.

42. The method according to claim 40, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

43. The method according to claim 35, wherein the cells of said population are substantially negative in the expression of the protein marker Sox-17, and also express at least one gene selected from the group consisting of: Hes-1 and Musashi-1.

44. The method according to claim 40, wherein the cells of said population are substantially negative in the expression of CD117, Oct4, Sox-17, and Tra2-54.

45. The method according to claim 44, wherein the cells of said population are substantially negative in the expression of cytokeratin protein.

46. The method according to claim 44, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

47. The method according to claim 44, wherein the cells of said population are substantially negative in the expression of the protein marker Sox-17, and also express at least one gene selected from the group consisting of: Hes-1 and Musashi-1.

48. The method according to claim 35, wherein the cells of said population are capable of propagating in vitro.

49. The method according to claim 35, wherein the cells of said population are capable of propagating in vitro under hypoxic conditions.

50. The method according to claim 35, wherein the cells of said population are capable of differentiating into cells displaying the characteristics of the β-cell lineage.

51. The method according to claim 35, wherein the cells of said population are capable of differentiating into a gut hormone-producing cell.

52. A method of treating a patient with diabetes mellitus or at risk of developing diabetes, comprising:
a. Isolating a population of amniotic fluid-derived cells from a donor, and
b. Transferring the cells into the patient.

53. The method according to claim 52, wherein the cells of said population are substantially negative in the expression of at least one protein marker selected from the group consisting of: CD117, Oct4, and Tra2-54.

54. The method according to claim 53, wherein the cells of said population are substantially positive for the expression of at least one protein marker selected from the group consisting of: HNF-1 beta, GATA-6, and Sox-17.

55. The method according to claim 53, wherein the cells of said population also express at least one gene selected from the group consisting of: HNF-3 beta, Hes-1, GATA-4 and Musashi-1.

56. The method according to claim 53, wherein the cells of said population are positive for the expression of at least one protein marker selected from the group consisting of: Liver Activator Protein, PDGF receptor β, AXL, bFGF, EGF receptor, Fas/TNFRSF6, GRO, GRO-alpha, ICAM-1, IL-1 alpha, Il-3, Il-6, Il-8, MIF, Osteoprotegerin, TIMP-2, and TRAIL R3.

57. The method according to claim 53, wherein the cells of said population are substantially negative in the expression of the protein marker Sox-17.

58. The method according to claim 57, wherein the cells of said population are substantially negative in the expression of cytokeratin protein.

59. The method according to claim 57, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

60. The method according to claim 52, wherein the cells of said population are substantially negative in the expression of the protein marker Sox-17, and also express at least one gene selected from the group consisting of: Hes-1 and Musashi-1.

61. The method according to claim 57, wherein the cells of said population are substantially negative in the expression of CD117, Oct4, Sox-17, and Tra2-54.

62. The method according to claim 61, wherein the cells of said population are substantially negative in the expression of cytokeratin protein.

63. The method according to claim 61, wherein the cells of said population are positive for the expression of at least one protein marker selected from the group consisting of: Liver Activator Protein, PDGF receptor β, bFGF, EGF receptor, GRO, GRO-alpha, ICAM-1, Il-3, Il-6, MIF, Osteoprotegerin, TIMP-2, and TRAIL R3.

64. The method according to claim 61, wherein the cells of said population are substantially negative in the expression of the protein marker Sox-17, and also express at least one gene selected from the group consisting of: Hes-1 and Musashi-1.

65. The method according to claim 52, wherein the cells of said population are differentiated into pancreatic hormone producing cells prior to the step of transferring into the patient.

66. The method according to claim 52, wherein the cells of said population differentiate into pancreatic hormone producing cells after the step of transferring into the patient.

67. The method according to claim 52, wherein the cells of said population are capable of propagating in vitro.

68. The method according to claim 52, wherein the cells of said population are capable of propagating in vitro under hypoxic conditions.

69. The method according to claim 52, wherein the cells of said population are capable of differentiating into cells displaying the characteristics of the β-cell lineage.

70. The method according to claim 52, wherein the cells of said population are capable of differentiating into a gut hormone-producing cell.
